SEQUENCE LISTING (35 sequences)

SEQ ID NO: 1 - protein, 172 aa, Homo sapiens
CDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 2 - protein, 172 aa, Homo sapiens
CDLPQNHGLLSRNTLVLLHQMRRISPFLCLKDRRDFRFPQEMVKGSQLQKAHVMSVLHEMLQQIFSLFHTERSSAAWNMELLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 3 - protein, 177 aa, Pan troglodytes
CDLPQNHGLLSRNTLVLLHQMRRISPFLCLKDRRDFRFPQEMVKGSQLQKAQVMSVLHEMLQQIFSLFHTERSSAAWNMTLLDQLHTGLHQQLQHLETCLLQVMGEGESAGAISSPALTLRRYFQGIRVYLKEKKYSDCAWEVVRMEIMKSLFLSTNMQERLRSKDRDLGSSRNDSH

SEQ ID NO: 4 - protein, 172 aa, Macaca fascicularis
CDLPQNHGLLSRNTLVLLHQMRRISPFLCLKDRRDFRFPQEMVEGSQLQKAQVMSVLHEMLQQIFSLFHTEHSSAAWNTTLLDHLHTGLHRQLEHLETCLVQVMREGESAGAIRSPALTLRRYFQGIRVYLKEKKYSDCAWVVVRMEIMKSLFLSTNMQERLKSKDGDLGSS

SEQ ID NO: 5 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 6 - protein, 166 aa, Homo sapiens
CDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 7 - protein, 166 aa, Homo sapiens
CDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 8 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNVDSILAVKKYFRRITLYLTEKKYSPCAWEVVRAEIMRSLSLSTNLQERLRRKE

SEQ ID NO: 9 - protein, 166 aa, Homo sapiens
CDLPQTHSLGNRRALILLAQMGRISPFSCLKDRHDFGFPQEEFDGNQFQKAQAISVLHEMIQQTFNLFSTKDSSATWEQSLLEKFSTELNQQLNDMEACVIQEVGVEETPLMNVDSILAVKKYFQRITLYLTEKKYSPCAWEVVRAEIMRSFSLSKIFQERLRRKE

SEQ ID NO: 10 - protein, 166 aa, Homo sapiens
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 11 - protein, 166 aa, Homo sapiens
CNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 12 - protein, 166 aa, Homo sapiens
CDLPQTHSLGNRRALILLAQMGRISPFSCLKDRPDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 13 - protein, 166 aa, Homo sapiens
CDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 14 - protein, 166 aa, Homo sapiens
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 15 - protein, 166 aa, Homo sapiens
CDLPQTHSLGNRRALILLAQMGRISHFSCLKDRHDFGFPEEEFDGHQFQKTQAISVLHEMIQQTFNLFSTEDSSAAWEQSLLEKFSTELYQQLNDLEACVIQEVGVEETPLMNVDSILAVRKYFQRITLYLTEKKYSPCAWEVVRAEIMRSLSFSTNLQKRLRRKD

SEQ ID NO: 16 - protein, 166 aa, Homo sapiens
CDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 17 - protein, 165 aa, Homo sapiens [sequence omitted]

SEQ ID NO: 18 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 19 - protein, 166 aa, Homo sapiens
CDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 20 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 21 - protein, 23 aa, synthetic construct
  REGION 1..23: note = Description of Artificial Sequence: Synthetic peptide
MALTFYLLVALVVLSYKSFSSLG

SEQ ID NO: 22 - protein, 23 aa, synthetic construct
  REGION 1..23: note = Description of Artificial Sequence: Synthetic peptide
MARSFSLLMVVLVLSYKSICSLG

SEQ ID NO: 23 - protein, 23 aa, synthetic construct
  REGION 1..23: note = Description of Artificial Sequence: Synthetic peptide
MALPFALLMALVVLSCKSSCSLD

SEQ ID NO: 24 - protein, 23 aa, synthetic construct
  REGION 1..23: note = Description of Artificial Sequence: Synthetic peptide
MALSFSLLMAVLVLSYKSICSLG

SEQ ID NO: 25 - protein, 23 aa, synthetic construct
  REGION 1..23: note = Description of Artificial Sequence: Synthetic peptide
MALTFALLVALLVLSCKSSCSVG

SEQ ID NO: 26 - protein, 557 aa, Homo sapiens
MMVVLLGATTLVLVAVAPWVLSAAAGGKNLKSPQKVEVDIIDDNFILRWNRSDESVGNVTFSFDYQKTGMDNWIKLSGCQNITSTKCNFSSLKLNVYEEIKLRIRAEKENTSSWYEVDSFTPFRKAQIGPPEVHLEAEDKAIVIHISPGTKDSVMWALDGLSFTYSLVIWKNSSGVEERIENIYSRHKIYKLSPETTYCLKVKAALLTSWKIGVYSPVHCIKTTVENELPPPENIEVSVQNQNYVLKWDYTYANMTFQVQWLHAFLKRNPGNHLYKWKQIPDCENVKTTQCVFPQNVFQKGIYLLRVQASDGNNTSFWSEEIKFDTEIQAFLLPPVFNIRSLSDSFHIYIGAPKQSGNTPVIQDYPLIYEIIFWENTSNAERKIIEKKTDVTVPNLKPLTVYCVKARAHTMDEKLNKSSVFSDAVCEKTKPGNTSKIWLIVGICIALFALPFVIYAAKVFLRCINYVFFPSLKPSSSIDEYFSEQPLKNLLLSTSEEQIEKCFIIENISTIATVEETNQTDEDHKKYSSQTSQDSGNYSNEDESESKTSEELQQDFV

SEQ ID NO: 27 - protein, 331 aa, Homo sapiens
MLLSQNAFIFRSLNLVLMVYISLVFGISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKIGGIITVFLIALVLTSTIVTLKWIGYICLRNSLPKVLRQGLAKGWNAVAIHRCSHNALQSETPELKQSSCLSFPSSWDYKRASLCPSD

SEQ ID NO: 28 - protein, 121 aa, synthetic construct
  REGION 1..121: note = Description of Artificial Sequence: Synthetic polypeptide
EVQLVQSGAEVKKPGESLKISCKGSGYSFTSYWIGWVRQMPGKGLEWMGIIDPSDSDTRYSPSFQGQVTISADKSISTAYLQWSSLKASDTAMYYCARHPGLNWAPDFDYWGQGTLVTVSS

SEQ ID NO: 29 - protein, 108 aa, synthetic construct
  REGION 1..108: note = Description of Artificial Sequence: Synthetic polypeptide
DIQMTQSPSSLSASVGDRVTITCRASQSIDNSYLNWYQQKPGKAPKLLIYGASSLQSGVPSRFSGSGSGTDFTLTISSLQPEDFATYYCQQGYDFPLTFGQGTKVEIK

SEQ ID NO: 30 - protein, 7 aa, synthetic construct
  REGION 1..7: note = Description of Artificial Sequence: Synthetic peptide
QSIDNSY

SEQ ID NO: 31 - skipped

SEQ ID NO: 32 - protein, 9 aa, synthetic construct
  REGION 1..9: note = Description of Artificial Sequence: Synthetic peptide
QQGYDFPLT

SEQ ID NO: 33 - protein, 8 aa, synthetic construct
  REGION 1..8: note = Description of Artificial Sequence: Synthetic peptide
GYSFTSYW

SEQ ID NO: 34 - protein, 8 aa, synthetic construct
  REGION 1..8: note = Description of Artificial Sequence: Synthetic peptide
IDPSDSDT

SEQ ID NO: 35 - protein, 14 aa, synthetic construct
  REGION 1..14: note = Description of Artificial Sequence: Synthetic peptide
ARHPGLNWAPDFDY

(19) United States
(12) Patent Application Publication
(10) Pub. No.: US 2025/0376516 A1
(43) Pub. Date: Dec. 11, 2025

(54) METHOD OF ADMINISTRATION OF AN ANTI-IFN-ALPHA/-OMEGA ANTIBODY

(71) Applicant: Janssen Biotech, Inc., Horsham, PA (US)

(72) Inventors: Marc Chevrier, Collegeville, PA (US); Jarrat Jordan, Norristown, PA (US)

(21) Appl. No.: 18/974,431

(22) Filed: Dec. 9, 2024

Related U.S. Application Data
(63) Continuation of application No. 16/839,249, filed on Apr. 3, 2020, now Pat. No. 12,202,892.
(60) Provisional application No. 62/829,317, filed on Apr. 4, 2019.

(51) Int. Cl. C07K 16/24; A61K 9/00; A61K 31/4706; A61K 31/519; A61K 31/573; A61K 39/00; A61K 47/12; A61K 47/26; A61P 37/02

(52) U.S. Cl. CPC C07K 16/249; A61K 47/12; A61K 47/26; A61P 37/02; A61K 9/0019; A61K 31/4706; A61K 31/519; A61K 31/573; A61K 2039/505; A61K 2039/54; A61K 2039/545

(57) ABSTRACT

Methods for administration of an anti-IFN-α/-ω antibody by subcutaneous or intravenous administration in a clinically proven safe amount are provided. Also provided are methods for clinically proven safe treatment of IFN-I mediated diseases, such as systemic lupus erythematosus (SLE), by subcutaneous or intravenous administration of an anti-IFN-α/-ω antibody.

### CROSS-REFERENCE TO RELATED APPLICATIONS

This application is a continuation of and claims the benefit of priority to U.S. patent application Ser. No. 16/839,249, filed 3 Apr. 2020, which claims the benefit of priority to U.S. Provisional Patent Application Ser. No. 62/829,317, filed 4 Apr. 2019, the entire contents of which are incorporated herein by reference in their entireties.

REFERENCE TO SEQUENCE LISTING SUBMITTED ELECTRONICALLY

This application contains a Sequence Listing, which is submitted electronically via Patent Center as an XML formatted sequence listing, with a file name “53359907AZD.xml”, creation date of Jun. 20, 2025 and having a size of about 38,846 bytes. The subject sequence listing submitted via Patent Center is part of the specification and is herein incorporated by reference in its entirety.

### FIELD OF THE INVENTION

The invention relates to methods of administration of an anti-IFN-α/-ω antibody in a clinically proven safe amount and methods of treating an IFN-I mediated disease by subcutaneous or intravenous administration of an anti-IFN-α/-ω antibody in a clinically proven safe amount.

### BACKGROUND OF THE INVENTION

Type I interferons (IFNs) (IFN-I) are a family of cytokines that signal through a ubiquitously expressed heterodimeric receptor IFNAR (heterodimer of IFNAR1 and IFNAR2) resulting in antiviral, antiproliferative and immunomodulatory effects. In humans, type I IFN is composed of at least 12 IFN-αprotein subtypes and 1 subtype each for IFN-β, IFN-ε, IFN-κ, and IFN-ω. IFN-I release occurs in response to both microbial and sterile ligands.

The IFN-I family of cytokines signal through the ubiquitously expressed heterodimeric IFN-αreceptor (IFNAR). Type I IFN can be induced by both microbial and endogenous factors (Elkon & Stone, Journal Interferon Cytokine Res., 31(11):803-812 (2011); Finke et al., Autoimmunity, 42(4):349-352 (2009)). IFN is rapidly produced in response to infectious agents, such as viruses, to help control infection. Microbial agents, endogenous ligands (e.g., necrotic cells), and immune complexes can trigger IFN-I production by activating both toll-like receptor (TLR)-dependent and TLR-independent pathways. The resulting IFN-I binds to the IFNAR resulting in the activation of antiviral, antiproliferative, and immunomodulatory genes. This so-called IFN-inducible gene signature has been observed in many autoimmune diseases.

Several immune-mediated inflammatory diseases or autoimmune diseases, such as lupus, including Systemic Lupus Erythematosus (SLE) and cutaneous lupus erythematosus (CLE), type I diabetes, psoriasis, Sjögren's disease, systemic sclerosis, rheumatoid arthritis, immune thrombocytopenia (ITP), Aicardi-Goutieres syndrome (AGS), myositis, common variable immune deficiency (CVID) and autoimmune thyroid disease are associated, at least in a sub-population of patients, with overexpression of IFN-inducible gene transcripts commonly called the IFN signature present in whole blood and/or tissue, or with elevated IFN-I.

SLE is a chronic autoimmune or immune-mediated inflammatory disease in which pathogenic T cell, B cell and innate immune responses result in inflammation and tissue damage across multiple organ systems. The disease displays a broad range of symptoms with heterogeneous clinical presentation and can include systemic, cutaneous, renal, musculoskeletal, neurological and hematological manifestations. SLE varies greatly in severity and is chronic, remitting or relapsing with flares of activity cycling with periods of improvement or remission that can last weeks, months, or years.

IFN-αis elevated in SLE patients and is believed to promote a loss of tolerance to self-antigens. IFN-αhas been shown to contribute to sustained dendritic cell activation and thus antigen presentation, and suppression of Treg function contributing to SLE. IFN-αalso induces BLyS expression, a target for the marketed SLE therapeutic BENLYSTA™. A number of polymorphisms associated with production or response to IFN-I have been identified and account for over half of confirmed polymorphisms associated with SLE (Ghodke-Puranik & Niewold, International journal of clinical rheumatology 8, doi: 10.2217/ijr.13.58 (2013)). Antibodies neutralizing various IFN-αsubtypes (pan-IFN-αantibodies) are being evaluated in clinical trials for SLE (see, for example, Int. Pat. Publ. No. WO02/066649, Int Pat. Publ. No. WO05/059106, Int. Pat. Publ. No. WO06/086586, Int. Pat. Publ. No. WO09/135861).

In addition to IFN-αs, IFN-ω can represent a key additional subclass of type 1 IFN that is overexpressed in SLE patients. SLE patients are known to produce autoantibodies to IFNs, and in two studies, the highest levels of nascent autoantibodies were against IFN-ω, implying that IFN-ω can be more prevalent in some SLE patients than other classes of IFN (McBride et al., Arthritis Rheum., 64(11):3666-3676 (2012); Petri et al., Arthritis Rheum., 65(4):1011-1021 (2013)). The anti-IFN-α antibodies currently in clinical trials (sifalimumab (MEDI-545), rontalizumab and AGS-009) do not neutralize IFN-ω. Clinical trial data with these antibodies indicate partial reduction of the type I IFN signature in patients after treatment with anti-IFN-α antibodies (Merrill et al., Ann Rheum Dis 70:1905-1913, 2011; Yao et al., Arthritis Rheum 60:1785-1796, 2009), and Phase 2 trial data with rontalizumab (a pan-anti-IFN-αantibody) indicated improvement in signs and symptoms of SLE, flare rates, and steroid burden at week 24 in a pre-specified biomarker defined group of Interferon Signature Metric (ISM)-Low moderate to severely active lupus subjects. No efficacy was seen in patients having higher levels of IFN-inducible gene expression pre-defined as ISM-High (Kalunian et al., 2012 ACR/ARHP Annual Meeting; Abstract #2622, 2012).

Current standard of care for SLE includes corticosteroids, antimalarial drugs, immunosuppressants or B cell modulators. These therapeutics can exhibit toxicity and other serious side effects and can not be suitable for treatment of all lupus patients. Thus, there is a need for additional therapeutic treatments for SLE and other IFN-I mediated diseases.

### BRIEF SUMMARY OF THE INVENTION

The invention relates to the clinically proven safe administration of an anti-IFN-α/-ω antibody to subjects, including for clinically proven safe treatment of an IFN-I mediated disease in a subject, such as systemic lupus erythematosus (SLE).

In one general aspect, the invention relates to a method of providing clinically proven safe administration of an anti-IFN-α/-ω antibody to a human subject in need thereof, comprising subcutaneously or intravenously administering to the subject a pharmaceutical composition comprising the antibody and a pharmaceutically acceptable carrier, wherein the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 33, 34, and 35, respectively, and the light chain variable region comprising light chain complementarity determining regions (LCDRs) LCDR1, LCDR2 and LCDR3 amino acid sequences of SEQ ID NOs: 30, 31, and 32, respectively, and wherein a total dosage of the antibody administered is 0.1 mg/kg to 20 mg/kg body weight of the subject per administration.

In one embodiment, the anti-IFN-α/-ω antibody comprises a heavy chain variable region (VH) having the amino acid sequence of SEQ ID NO: 28 and a light chain variable region (VL) having the amino acid sequences of SEQ ID NO: 29.

In one embodiment, the pharmaceutical composition is administered intravenously. In such embodiments, the total dosage of the anti-IFN-α/-ω antibody administered per administration is 0.1 mg/kg, 0.3 mg/kg, 1 mg/kg, 3 mg/kg, 10 mg/kg, 15 mg/kg or 20 mg/kg body weight of the subject, or any dosage in between.

In one embodiment, the pharmaceutical composition is administered subcutaneously. In such embodiments, the total dosage of the anti-IFN-α/-ω antibody administered per administration is 0.1 mg/kg, 0.3 mg/kg, 0.5 mg/kg, 0.7 mg/kg, 1 mg/kg, 1.2 mg/kg, 1.5 mg/kg, 1.7 mg/kg, 2 mg/kg, 2.3 mg/kg or 2.5 mg/kg body weight of the subject, or any dosage in between.

In one embodiment, the administration of the pharmaceutical composition achieves, in the plasma of the subject, at least one parameter selected from: (i) an area under the concentration time curve (AUC)₍₀₋ₜ₎ of about 50 μg.day/mL to about 7000 μg.day/mL, and (ii) a maximum concentration observed (Cₘₐₓ) of about 5 μg/mL to about 500 μg/mL.

In one embodiment, the administration of the anti-IFN-α/-ω antibody does not result in production of antibodies against the anti-IFN-α/-ω antibody in the subject.

In another general aspect, the invention relates to a method of providing clinically proven safe treatment of an IFN-I mediated disease in a human subject in need thereof, the method comprising subcutaneously or intravenously administering to the subject a pharmaceutical composition comprising the antibody and a pharmaceutically acceptable carrier, wherein the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 33, 34, and 35, respectively, and the light chain variable region comprising light chain complementarity determining regions (LCDRs) LCDR1, LCDR2 and LCDR3 amino acid sequences of SEQ ID NOs: 30, 31, and 32, respectively, and wherein a total dosage of the antibody administered is 0.1mg/kg to 20 mg/kg body weight of the subject per administration.

In one embodiment, the IFN-I mediated disease is selected from systemic lupus erythematosus (SLE), type I diabetes, psoriasis, primary Sjögren's disease, systemic sclerosis, rheumatoid arthritis, transplant rejection, dermatomyositis, polymyositis, Aicardi-Goutières syndrome, Sting associated vasculopathy with onset in infancy (SAVI) or chronic atypical neutrophilic dermatosis with lipodystrophy and elevated temperature syndrome (CANDLE). Preferably, the disease is systemic lupus erythematosus (SLE), and more preferably, mild to moderate SLE.

In one embodiment, the anti-IFN-α/-ω antibody comprises a heavy chain variable region (VH) having the amino acid sequence of SEQ ID NO: 28 and a light chain variable region (VL) having the amino acid sequences of SEQ ID NO: 29.

In one embodiment, the anti-IFN-a/-antibody is an IgG1 isotype. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The anti-IFN-α/-ω antibody may be of any IgG1 allotype, such as Glm17, G1m3, Glm1, G1m2, G1m27 or G1m28.

In one embodiment, the pharmaceutical composition is administered to the human subject intravenously for no less than 30 minutes in a total dosage of the anti-IFN-α/-ω antibody administered of 10 mg/kg body weight of the subject per administration, preferably the pharmaceutical composition is intravenously administered to the human subject repeatedly, more preferably once every two weeks.

In one embodiment, the administration of the pharmaceutical composition achieves, in the plasma of the subject, at least one parameter selected from: (i) an area under the concentration time curve (AUC)₍₀₋₁₄ₐ₎ of about 1000 μg.day/mL to about 3500 μg.day/mL, and (ii) a maximum concentration observed (Cₘₐₓ) of about 120 μg/mL to about 400 μg/mL.

In one embodiment, the human subject, by day 100 after the administration of the pharmaceutical composition, has at least one of clinical responses selected from i) a reduction in the Systemic Lupus Erythematosus Responder Index (SRI); ii) no new British Isles Lupus Assessment Group (BILAG) A or 2B shifts; iii) a reduction in the Systemic Lupus Erythematosus Disease Activity Index 2000 (SLEDAI-2K) from baseline; iv) a reduction in the Systemic Lupus Erythematosus 2000 Responder Index-50 (S2K RI-50) from baseline; and v) a reduction in the Physician's Global Assessment of Disease Activity (PGA) from baseline.

In one embodiment, the human subject achieves a steady-state condition of the antibody within 40-50 days after administration.

In one embodiment, the administration of the anti-IFN-α/-ω antibody does not result in a treatment emergent adverse event (TEAE) related to a malignancy or anaphylactic or serum sickness-type reaction in the subject.

In another general aspect, the method of the invention further comprises:
a. assaying gene expression of one or more genes DHX58, EIF2AK2, HERC5, IFI44, IFI44L, IFI6, IRF7, PARP9, PLSCR1 and SAMD9L in a biological sample of the human subject; and
b. identifying the human subject as responsive to treatment of the antibody prior to administering to the subject the pharmaceutical composition comprising the antibody and the pharmaceutically acceptable carrier.

The details of one or more embodiments of the invention are set forth in the description below. Other features and advantages will be apparent from the following detailed description, and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows a diagrammatic representation of the study design of a clinical study using an anti-IFN-α/-ω antibody (ANTIBODY A) according to an embodiment of the application.

FIG. 2 shows the disposition of subjects in Part A of the study.

FIG. 3 shows the disposition of subjects in Part B of the study.

FIG. 4 demonstrates mean (SD) serum concentration-time profiles of ANTIBODY A after single intravenous infusion of ANTIBODY A at doses ranging from 0.3 to 15 mg/kg in healthy subjects (Cohorts 1-5).

FIG. 5 demonstrates mean (SD) serum concentration-time profiles of ANTIBODY A after a single intravenous infusion of ANTIBODY A 1.0 mg/kg (Cohort 2) or after a single subcutaneous injection of ANTIBODY A 1.0 mg/kg (Cohort 6) in healthy subjects.

FIG. 6 demonstrates mean (SD) serum concentration-time profiles of ANTIBODY A after multiple intravenous infusions of 10 mg/kg ANTIBODY A in subjects with mild to moderate systemic lupus erythematosus.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “subject” refers to a mammalian subject, preferably human, diagnosed with or suspected of having an IFN-I mediated disease, whom will be or has been administered an anti-IFN-«/-ω antibody according to a method of the invention. Diagnosis of an IFN-I mediated disease can be done by a clinician according to clinical diagnostic testing, physical examination of the subject, or any other accepted method for diagnosing a subject with a particular disease.

A used herein, “a subject suspected of having an IFN-I mediated disease” is a subject that presents signs or symptoms indicative of an IFN-I mediated disease that are discernable to a clinician and/or the subject, but whose suspected diagnosis has not been confirmed by clinical diagnostic testing, physical examination of the subject, or other accepted method for diagnosing a subject with the suspected IFN-I mediated disease.

Examples of IFN-I mediated diseases include, but are not limited to, Systemic Lupus Erythematosus (SLE), cutaneous lupus erythematosus (CLE), type I diabetes, psoriasis, Sjögren's disease, systemic sclerosis, rheumatoid arthritis, immune thrombocytopenia (ITP), Aicardi-Goutieres syndrome (AGS), myositis, common variable immune deficiency (CVID), autoimmune thyroid disease, transplant rejection, dermatomyositis, polymyositis, Sting associated vasculopathy with onset in infancy (SAVI), and chronic atypical neutrophilic dermatosis with lipodystrophy and elevated temperature syndrome (CANDLE).

The term “type I interferon” or “IFN-I” refers to all native subtypes of human interferon-a and one subtype of interferon-β, interferon-ε, interferon-ω and interferon-κ which bind to a common interferon receptor IFNAR.

The term “interferon-α” (IFN-α) as used herein refers to all native subtypes of human alpha interferons. Native IFN-αconsists of at least 12 closely related protein subtypes encoded by distinct genes with a high degree of structural homology (Weissmann and Weber, Prog Nucl Acid Res Mol Biol., 33:251, 1986; Roberts et al., J Interferon Cytokine Res. 18:805-816, 1998). Nomenclature for human interferons is found at: http://www_genenames_org/genefamilies/_IFN. Table 1 below shows the sequences of the IFN-αsubtypes used herein, in addition to other Type I IFNs.

The term IFN-@ as used herein refers to human IFN-@ having the amino acid sequence shown in SEQ ID NO: 1 and UniProt accession number P05000. Human IFN-ω also includes the variant of SEQ ID NO: 2 having a threonine to glutamic acid substitution at position 80 (T80).

The IFN-αsubtypes and IFN-o can also be produced by recombinant expression using standard methods. Exemplary signal sequences that can be used for directing secretion are shown in SEQ ID NOs: 21-25.

As used herein the term “IFNAR” refers to the well-known interferon receptor which is a heterodimer of IFNAR1 and IFNAR2. IFNAR1 and IFNAR2 protein sequences are shown in SEQ ID NOs: 26 and 27, respectively. IFNAR1 mature extracellular domain spans residues 28-436 of SEQ ID NO: 26 and IFNAR2 mature extracellular domain spans residues 27-243 of SEQ ID NO: 27.

TABLE 1

| | | GenBank | |
|---|---|---|---|
| | Alternative | Accession Number | SEQ ID |
| IFN Protein | Name | Adopted | NO: |
| IFN-αA | IFN-α2s | V00549 | 5 |
| IFN-αB2 | IFN-α8 | X03125 | 6 |
| IFN-αC | IFN-α10 | NM_002171.1 | 7 |
| IFN-αD | Val114 IFN-α1 | V00538 | 8 |
| IFN-αF | IFN-α21 | V00540 | 9 |
| IFN-αG | IFN-α5 | X02956 | 10 |
| IFN-αH2 | IFN-α14 | X02959 | 11 |
| IFN-αI | IFN-α17 | V00532 | 12 |
| IFN-αJ1 | IFN-α7 | X02960 | 13 |
| IFN-αK | IFN-α6 | X02958 | 14 |
| IFN-α4b | IFN-α4 | X02955 | 15 |
| IFN-αWA | IFN-α16 | X02957 | 16 |
| IFN-α2 | IFN-α2b | V00548, | 17 |
| | | NM_00605.2 | |
| IFN-α1 | Ala114 IFN-αD | J00210 | 18 |
| IFN-α4a | IFN-αM1 | NM_021068 | 19 |
| IFN-β | | V00534 | 20 |
| IFN-ω | | NM_002177.1 | 1 |
| IFN-ω T80E | | | 2 |
| Chimp IFN-ω | | XM_528554.1 | 3 |
| Cyno IFN-ω | | NA | 4 |

As used herein, an “an anti-IFN-α/-ω antibody,” refers to an engineered fully human monoclonal antibody (mAb) of the IgG1κ subtype, or antigen binding fragment thereof, that binds and neutralizes at least 10 human interferon alpha (IFN-α) subtypes at an affinity of 1×10⁻¹⁰ M or less and further binds and neutralizes human interferon omega (IFN-ω) with high affinity. Preferably, the anti-IFN--α/-ω antibody does not bind or neutralize interferon beta (IFN-β). Examples of anti-IFN-α/-ω antibody or antigen binding fragment thereof useful for the invention include, but are not limited to, IFWM3405, IFWM3442, IFWM3525, IFWM3423, IFWM3444, IFWM3421, or other anti-IFN-α/-ω antibodies or fragments thereof described in U.S. Pat. No. 10,208,113, the content of which is herein incorporated by reference in its entirety. In a preferred embodiment, the anti-IFN-α/-ω antibody neutralizes at least 11 out of the 12 IFN alphas. In another preferred embodiment, the anti-IFN-α/-ω antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 33, 34, and 35, respectively, and the light chain variable region comprising light chain complementarity determining regions (LCDRs) LCDR1, LCDR2 and LCDR3 amino acid sequences of SEQ ID NOs: 30, 31, and 32, respectively. In one embodiment, the anti-IFN-α/-ω antibody comprises a heavy chain variable region (VH) having the amino acid sequence of SEQ ID NO: 28 and a light chain variable region (VL) having the amino acid sequences of SEQ ID NO: 29. In a preferred embodiment, the anti-IFN-α/-ω antibody is a humanized or fully human immunoglobulin.

Anti-IFN-α/-ω antibodies can be prepared by any method known in the art in view of the present disclosure for preparing monoclonal antibodies including, but not limited to, hybridoma production. For example, anti-IFN--α/-ω antibodies can be produced in a mammalian cell line (e.g., Chinese Hamster Ovary (CHO) cell line) using recombinant DNA technology. In particular, methods of producing anti-IFN-α/-ω antibodies useful for the invention are further described in, e.g., U.S. Pat. No. 10,208,113, which is herein incorporated by reference.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IFN-α/-ω. For example, antibody fragments capable of binding to INF-α/-ω or portions thereof, including, but not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques or can be prepared as a contiguous protein using genetic engineering techniques.

As used herein, the term “human antibody” refers to an antibody in which substantially every part of the protein (e.g., CDR, framework, CL, CH domains (e.g., CH1, CH2, CH3), hinge, (VL, VH)) is substantially non-immunogenic in humans, with only minor sequence changes or variations. A “human antibody” can also be an antibody that is derived from or closely matches human germline immunoglobulin sequences. Human antibodies can include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

The term “safe,” as it relates to a dose, dosage regimen, treatment or method with an anti-IFN-α/-ω antibody refers to a favorable risk: benefit ratio with an acceptable frequency and/or acceptable severity of treatment-emergent adverse events (referred to as AEs or TEAEs) compared to the standard of care or to another comparator in accordance with the Federal Food, Drug, and Cosmetic Act, as amended (secs. 201-902, 52 Stat. 1040 et seq., as amended; 21 U.S.C. §§ 321-392). In particular, safe as it relates to a dose, dosage regimen, or treatment with an anti-IFN-α/-ω antibody of the present invention refers to with an acceptable frequency and/or acceptable severity of adverse events associated with administration of the antibody if attribution is considered to be possible, probable, or very likely due to the use of the anti-IFN-α/-ω antibody. Safety is often measured by toxicity testing to determine the highest tolerable dose or the optimal dose of an active pharmaceutical ingredient needed to achieve the desired benefit. Studies that look at safety also seek to identify any potential adverse effects that can result from exposure to the drug.

As used herein, unless otherwise noted, the term “clinically proven” (used independently or to modify the term “safe”) shall mean that it has been proven by a clinical study in human subjects wherein the clinical study has met the approval standards of U.S. Food and Drug Administration, European Medicines Evaluation Agency (EMEA), or a corresponding national regulatory agency. In one embodiment of the application, the clinical study is a phase 1, randomized, double-blind, placebo-controlled, single ascending dose study of an anti-IFN-α/-ω antibody in healthy human subjects. Preferably, the clinical study further comprises a multiple dose study of an anti-IFN-α/-ω antibody in human subjects with mild to moderate systemic lupus erythematosus.

As used herein, the phrases “adverse event,” “treatment-emergent adverse event,” and “adverse reaction” mean any harm, unfavorable, unintended or undesired sign or outcome associated with or caused by administration of a pharmaceutical composition or therapeutic. However, abnormal values or observations are not reported as adverse events unless considered clinically significant by the investigator. As used herein, when referring to an adverse event, “clinically apparent” means clinically significant as determined by a medical doctor or an investigator using standard acceptable to those of ordinary skill in the art. When the harm or undesired outcome of adverse events reaches such a level of severity, a regulatory agency can deem the pharmaceutical composition or therapeutic unacceptable for the proposed use.

Examples of adverse events or reactions when used in the context of subcutaneous or intravenous administration of an anti-IFN-α/-ω antibody include, but are not limited to, infections and infestations, such as rhinitis, herpes zoster, and myringitis bullosa; respiratory, thoracic and mediastinal disorders, such as cough, throat irritation, and oropharyngeal pain; gastrointestinal disorders, such as diarrhea and flatulence; nervous system disorders, such as headache and dizziness; blood and lymphatic system disorders, such as anaemia and lymphadenopathy; back pain, premature labour, infusion reactions, local injection site reactivity, malignancy and no anaphylactic or serum sickness-type reactions.

As used herein, “treatment” or “treat” refers to therapeutic treatment. Individuals in need of treatment include those subjects diagnosed with the disorder or a symptom of the disorder. Subjects that can be treated also include those prone to or susceptible to have the disorder, of those in which the disorder is to be prevented. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. Beneficial clinical result includes, in a subject who has received treatment, for example reduced proliferation of B cells or dendritic cells, reduction of inflammatory cytokines, adhesion molecules, proteases, immunoglobulins, combinations thereof, increased production of anti-inflammatory proteins, a reduction in the number of autoreactive cells, an increase in immune tolerance, inhibition of autoreactive cell survival, and/or a decrease in one or more symptoms mediated by IFN-I. Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like.

The terms “efficacy” and “effective” as used herein in the context of a dose, dosage regimen, treatment or method refer to the effectiveness of a particular dose, dosage or treatment regimen. Efficacy can be measured based on change in the course of the disease in response to an agent of the present invention. For example, an anti-IFN-α/-ω antibody useful for the present invention is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, an anti-IFN-α/-ω antibody of the present invention can be administered to achieve an improvement in a subject's condition related to SLE.

Improvement on SLE can be evaluated by at least one of the following clinical response assessments: i) a reduction in the Systemic Lupus Erythematosus Responder Index (SRI), such as by 1 point, 2 points, 3 points, 4 points, 5 points, or 6 points,; ii) a shift in British Isles Lupus Assessment Group (BILAG); iii) a reduction in the Systemic Lupus Erythematosus Disease Activity Index 2000 (SLEDAI-2K) from baseline; iv) a reduction in the Systemic Lupus Erythematosus 2000 Responder Index-50 (S2K RI-50) from baseline; v) a reduction in the Physician's Global Assessment of Disease Activity (PGA) from baseline; vi) the change and percent change from baseline in Cutaneous Lupus Erythematosus Disease Area and Severity Index (CLASI) activity and damage scores; vii) the change and percent change from baseline in norm-based scores of SF-36, SF-36 physical and mental component summary scores; and viii) the change and percent change from baseline in EuroQol-5 dimensions-5 levels (EQ-5D-5L) scale.

In addition, joint assessment is used to evaluate the clinical response, e.g., the change from baseline in the number of joints with pain and signs of inflammation over time for subjects. Each of the joints are evaluated for tenderness and swelling (hips excluded for swelling) at all specified visits by an assessor.

In some embodiments, before subject to a treatment according to an embodiment of the application, a human subject having mild to moderate SLE is defined by:
1) Systemic Lupus International Collaborating Clinics (SLICC) criteria for diagnosis of lupus with at least 1 Systemic Lupus Erythematosus Disease Activity Index 2000 (SLEDAI-2K)-defined non-serologic clinical activity within 3 months prior to treatment, and
2) Serologically positive within 2 months prior to:
   a) a positive anti-nuclear antibody (ANA) titer of ≥1:80, or
   b) a positive anti-double stranded deoxyribonucleic acid test (note: if Anti-dsDNA is done by enzyme-linked immunosorbent assay, a test 2× upper limit of normal is required to be considered unequivocally positive) or
   c) a positive anti-Smith antibody and/or positive anti-ribonucleoprotein (RNP) antibody and/or anti-Ro antibody, and in addition to at least one of the above:
   d) a positive lupus IFN profile score during screening.

The responsiveness of a subject to a treatment can be measured by an index of disease activity, clinical symptoms or by any other measure of disease activity. As used herein, a “patient not responsive or poorly responsive to a treatment” refers to a patient who has no or minimal improvement after the treatment.

As used herein, a dosage amount of an anti-IFN-α/-ω antibody in “mg/kg” refers to the amount of the anti-IFN-α/-ω antibody in milligrams per kilogram of the body weight of a subject to be administered with the antibody.

In one general aspect, the invention relates to a method of providing clinically proven safe subcutaneous and/or intravenous administration of an anti-IFN-α/-ω antibody to a human subject in need thereof. Preferably, the subject is diagnosed with or suspected of having an IFN-I mediated disease, in which the IFN-I levels are elevated. There is an association between elevated levels of type I interferons (IFN-I) and several human autoimmune diseases such as systemic sclerosis, Sjögren's syndrome, type I diabetes and autoimmune thyroid disease, and most notably, systemic lupus erythematosus (SLE). Examples of an IFN-mediated disease with which a subject to be administered an anti-IFN-α/-ω antibody or antigen binding fragment thereof according methods of the invention can be diagnosed with or suspected of having include, but are not limited to, an autoimmune disease, such as systemic lupus erythematosus (SLE), type I diabetes, psoriasis, primary Sjögren's disease, systemic sclerosis, rheumatoid arthritis, transplant rejection, dermatomyositis, polymyositis, Aicardi-Goutières syndrome, Sting associated vasculopathy with onset in infancy (SAVI) or chronic atypical neutrophilic dermatosis with lipodystrophy and elevated temperature syndrome (CANDLE).

In one preferred embodiment, the subject is diagnosed with or suspected of having SLE. SLE can target many organs and tissues, including the skin, blood vessels, muscles, kidneys and lungs. Preclinical and clinical data support a role for IFN-αand IFN-ω in the initiation and progression of SLE.

In one embodiment, a method of providing clinically proven safe administration of an anti-IFN-α/-ω antibody to a subject and/or safe treatment of an IFN-mediated disease, preferably SLE in a human subject, comprises subcutaneously or intravenously administering to the subject a pharmaceutical composition comprising an anti-IFN-α/-ω antibody and a pharmaceutically acceptable carrier, wherein a total dosage of the anti-IFN-α/-ω antibody administered is 0.1 mg/kg to 20 mg/kg body weight of the subject per administration.

In one embodiment, the pharmaceutical composition is administered subcutaneously. Subcutaneous administration refers to administration under the skin, in which a drug or therapeutic is injected into the tissue layer between the skin and muscle. Medication administered via subcutaneous administration is usually absorbed more slowly than if injected into a vein. When administration of an anti-IFN-α/-ω antibody is via subcutaneous injection, the total dosage of the an anti-IFN-α/-ω antibody to be administered to the subject per administration can be administered in a single subcutaneous injection, or in multiple subcutaneous injections, such as 1, 2, 3, 4, 5, or more subcutaneous injections.

In another embodiment, the pharmaceutical composition is administered intravenously. Intravenous administration refers to administration directly into a vein. Intravenous administration can be via injection (e.g., with a syringe at higher pressures) or via infusion (e.g., using the pressure supplied by gravity). Intravenous administration is typically the quickest method for delivering a drug or therapeutic throughout the body, because the drug or therapeutic is carried by circulation. When administration of an anti-IFN-α/-ω antibody is via intravenous administration, administration can be by intravenous infusion or injection, and is preferably via infusion. For example, the total dosage of an anti-IFN-α/-ω antibody to be administered to the subject per administration can be administered by intravenous infusion over a period of time, such as about 30 minutes to 180 minutes, preferably 60 minutes to 120 minutes, such as 30 minutes, 60 minutes, 90 minutes, 120 minutes, 150 minutes, or 180 minutes, or any duration in between.

The total dosage of an anti-IFN-α/-ω antibody per administration is selected so as to provide safe administration and/or safe treatment by subcutaneous or intravenous administration as determined in clinical trials. According to embodiments of the invention, when the pharmaceutical composition is administered intravenously, a total dosage of the anti-IFN-α/-ω antibody administered per administration is, for example, 0.1 mg/kg, 0.3 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, or any dosage in between. When the pharmaceutical composition is administered subcutaneously, a total dosage of the anti-IFN-α/-ω antibody administered per administration is, for example, 0.1 mg/kg, 0.3 mg/kg, 0.5 mg/kg, 0.7 mg/kg, 1 mg/kg, 1.2 mg/kg, 1.5 mg/kg, 1.7 mg/kg, 2 mg/kg, 2.3 mg/kg, 2.5 mg/kg, or any dosage in between.

The total dosage of the anti-IFN-α/-ω antibody can be administered once per day, once per week, once per two weeks, once per month, once every six months, etc. for a period of one day, one week, one month, six months, 1 year, 2 years or longer. For example, a total dosage of 0.1 mg/kg to 2.5 mg/kg of the anti-IFN-α/-ω antibody can be administered per administration (e.g., once per day for at least one day) by a single subcutaneous injection, or multiple subcutaneous injections (e.g., 2 to 5 injections) at substantially the same time, i.e., over a time of period of 0 minutes to 1 hour, such as 5 minutes, 10 minutes, 15 minutes, 20 minutes, 25 minutes, 30 minutes, 35 minutes, 40 minutes, 45 minutes, 50 minutes, 55 minutes, or 1 hour. Alternatively, a total dosage of 0.3 mg/kg to 20 mg/kg of the anti-IFN-α/-ω antibody can be administered per administration (e.g., once per day for at least one day) by intravenous infusion over a time period of about 30 minutes to 3 hours, preferably 60 minutes to 120 minutes. Multiple administrations of the anti-IFN-α/-ω antibody, each at a total dosage of 0.3 mg/kg to 20 mg/kg, can be administered to a subject in need thereof.

Pharmaceutical compositions suitable for use in the methods of the invention are formulated for subcutaneous administration or intravenous administration. Examples of formulations suitable for subcutaneous and/or intravenous administration include, but are not limited to, solutions, suspensions, emulsions, and dry products that can be dissolved or suspended in a pharmaceutically acceptable carrier for injection or infusion. In a preferred embodiment, a pharmaceutical composition comprising an anti-IFN-α/-ω antibody for use in the methods of the invention is formulated as a solution.

A concentration of an anti-IFN-α/-ω antibody included in pharmaceutical compositions used in the invention can vary. Typically, the concentration of the anti-IFN-α/-ω antibody is 1 mg/mL to 100 mg/mL, such as 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL, or any concentration in between. Preferably, the concentration of the anti-IFN-α/-ω antibody is 40 mg/mL to 60 mg/mL, for instance 50 mg/mL.

Pharmaceutical compositions for use in the invention further comprise one or more pharmaceutically acceptable carriers, such as those widely employed in the art of drug manufacturing, and particularly antibody drug manufacturing. As used herein, the term “carrier” refers to any excipient, diluent, buffer, stabilizer, or other material well known in the art for pharmaceutical formulations. Pharmaceutically acceptable carriers in particular are non-toxic and should not interfere with the efficacy of the active ingredient. The pharmaceutically acceptable carriers include excipients and/or additives suitable for use in the pharmaceutical compositions known in the art, e.g., as listed in “Remington: The Science & Practice of Pharmacy”, 19th ed., Williams & Williams, (1995), and in the “Physician's Desk Reference”. 52nd ed., Medical Economics, Montvale, N.J. (1998), the disclosures of which are entirely incorporated herein by reference.

According to embodiments of the invention, a pharmaceutical composition for use in the invention comprises an anti-IFN-α/-ω antibody and a pharmaceutically acceptable carrier. In some embodiments, the pharmaceutically acceptable carrier comprises one or more carbohydrates, such as lactose, maltose, sucrose, one or more surfactants, such as polysorbate 20, polysorbate 80, and one or more acids and/or salts, such as lactic acid, sodium lactate, acetic acid, sodium acetate. Preferably, the pharmaceutical composition has a pH of 5 to 6, such as a pH of 5.0, 5.1, 5.2, 5.3, 5.4, 5.5, 5.6, 5.7, 5.8, 5.9, or 6.0, or any value in between.

In some embodiments, a pharmaceutical composition for use in the invention comprises sucrose, lactose, and/or maltose at a concentration of 1% to 10% weight by volume (w/v), such as 5% to 10% (w/v). For example, the concentration of sucrose, lactose, and/or maltose can be 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v), or any concentration in between.

In some embodiments, a pharmaceutical composition for use in the invention comprises polysorbate 20 (PS20) and/or polysorbate 80 (PS80) at a concentration of 0.01% (w/v) to 0.1% (w/v) or 0.02% (w/v) to 0.08% (w/v). For example, the concentration of polysorbate 20 and/or polysorbate 80 can be 0.01%, 0.02%, 0.03%, 0.04%, 0.05%, 0.06%, 0.07%, 0.08%, 0.09% or 0.1% (w/v), or any concentration in between.

In some embodiments, a pharmaceutical composition for use in the invention comprises lactic acid, sodium lactate, acetic acid or sodium acetate at a concentration of 10 mM to 20 mM based on the amount of lactate or acetate, respectively. For example, the concentration of lactate or acetate can be 10 mM, 11 mM, 12 mM, 13 mM, 14 mM, 15 mM, 16 mM, 17 mM, 18 mM, 19 mM, 20 mM, or any concentration in between.

Pharmaceutical compositions comprising an anti-IFN-α/-ω antibody for use in the invention can be prepared by any method known in the art in view of the present disclosure. For example, an anti-IFN-α/-ω antibody can be mixed with one or more pharmaceutically acceptable carriers to obtain a solution. The solution can be stored as a frozen liquid at a controlled temperature ranging from −40° C.±10° C. to −70° C.±20° C. and under protection from light exposure in an appropriate vial until administered to the subject.

According to embodiments of the invention, a variety of factors can be analyzed to determine by clinical trials such as those described herein whether a particular dosage of the anti-IFN-α/-ω antibody provides for safe subcutaneous and/or intravenous administration. For example, safety of a certain dosage of subcutaneously and/or intravenously administered anti-IFN-α/-ω antibody can be assessed by immunogenicity studies (e.g., measuring the production of antibodies to the anti-IFN-α/-ω antibody); by pharmacokinetic studies (e.g., an area under the concentration time curve (AUC), and a maximum concentration observed (Cₘₐₓ). The safety of subcutaneously and/or intravenously administered anti-IFN-α/-ω antibody can also be monitored by physical examination of the subject; observation of local injection site reactions, systemic injection related reactions, and other allergic reactions; electrocardiograms; clinical laboratory tests; vital signs; and monitoring of other adverse events, such as thromboembolic events.

In some embodiments, clinically proven safe administration of an anti-IFN-α/-ω antibody and/or clinically proven safe treatment of an autoimmune disease is determined by measuring amounts of antibodies to the administered anti-IFN-α/-ω antibody in a sample obtained from a subject. The amounts of antibodies to the anti-IFN-α/-ω antibody can be measured by any method known in the art in view of the present disclosure, e.g., ELISA.

In some embodiments, clinically proven safe administration of an anti-IFN-α/-ω antibody and/or clinically proven safe treatment of an IFN-mediated disease is determined by assessing pharmacokinetic (PK) parameters, such as an area under the concentration time curve (AUC), and a maximum concentration observed (Cₘₐₓ), of the anti-IFN-α/-ω antibody in the serum of the subject. Serum samples are analyzed to determine concentrations of the anti-IFN-α/-ω antibody by any method known in the art in view of the present disclosure. The pharmacokinetic parameters are then analyzed, for example by non-compartment analysis (NCA), to calculate pharmacokinetic parameters, such as AUC, Cₘₐₓ, terminal half-life (T_{1/2}), total systemic clearance after intravenous administration (CL), volume of distribution at terminal phase (V_{z}), total systemic clearance over bioavailability (CL/F), and volume of distribution at terminal phase over bioavailability (V_{z}/F).

In some embodiments, clinically proven safe administration of an anti-IFN-α/-ω antibody achieves AUC₍₀₋ₜ₎ (AUC versus time curve from time 0 to the time corresponding to the last quantifiable serum concentration) about 50 μg.day/mL to about 7000 μg.day/mL. For example, the AUC₍₀₋ₜ₎ can be 50 μg.day/mL, 100 μg.day/mL, 500 μg.day/mL, 1000 μg.day/mL, 2000 μg.day/mL, 3000 μg.day/mL, 4000 μg.day/mL, 5000 μg.day/mL, 6000 μg.day/mL, 7000 μg.day/mL, or any number in between.

In some embodiments, clinically proven safe administration of an anti-IFN-α/-ω antibody achieves a maximum concentration observed (Cₘₐₓ) of about 5 μg/mL to about 500 μg/mL. For example, the Cₘₐₓ can be about 5 μg/mL, 10 μg/mL, 50 μg/mL, 100 μg/mL, 200 μg/mL, 300 μg/mL, 400 μg/mL, 500 μg/mL, or any number in between.

In some embodiments, clinically proven safe treatment of an IFN-I mediated disease with administration of an anti-IFN-α/-ω antibody achieves AUC₍₀₋ₜ₎ (AUC versus time curve from time 0 to the time corresponding to the last quantifiable serum concentration) about 1000 μg.day/mL to about 3500 μg.day/mL. For example, the AUC₍₀₋ₜ₎ can be 1000 μg.day/mL, 1500 μg.day/mL, 2000 μg.day/mL, 2500 μg.day/mL, 3000 μg.day/mL, 3500 μg.day/mL, or any number in between.

In some embodiments, clinically proven safe treatment of an IFN-mediated disease with administration of an anti-IFN-α/-ω antibody achieves a maximum concentration observed (Cₘₐₓ) of about 120 μg/mL to about 400 μg/mL. For example, the Cₘₐₓ can be about 120 μg/mL, 150 μg/mL, 200 μg/mL, 250 μg/mL, 300 μg/mL, 350 μg/mL, 400 μg/mL, or any number in between.

In another general aspect, the method of the invention further comprises a method of diagnosing a subject having an IFN-I mediated disease that is responsive to treatment of an anti-IFN-α/-ω antibody, comprising:
a. assaying gene expression of one or more genes DHX58, EIF2AK2, HERC5, IFI44, IFI44L, IFI6, IRF7, PARP9, PLSCR1 and SAMD9L in a biological sample of the human subject; and
b. identifying the human subject as responsive to treatment of the antibody prior to administering to the subject the pharmaceutical composition comprising the antibody and the pharmaceutically acceptable carrier.

In particular, this diagnosing method is further described in, e.g., U.S. Provisional Patent Application No. 62/751,019, the contents of which are herein incorporated by reference in their entireties.

### EMBODIMENTS

The invention provides also the following non-limiting embodiments.

Embodiment 1 is a method of administration of an anti-IFN-α/-ω antibody or antigen binding fragment thereof to a human subject in need thereof in a clinically proven safe amount, comprising subcutaneously or intravenously administering to the human subject a pharmaceutical composition comprising the antibody and a pharmaceutically acceptable carrier, wherein a total dosage of the antibody administered is 0.1 mg/kg to 20 mg/kg body weight of the subject per administration.

Embodiment 1a is the method of embodiment 1, wherein the anti-IFN-α/-ω antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 33, 34, and 35, respectively, and the light chain variable region comprising light chain complementarity determining regions (LCDRs) LCDR1, LCDR2 and LCDR3 amino acid sequences of SEQ ID NOs: 30, 31, and 32, respectively.

Embodiment 1b is the method of embodiment 1 or la, wherein the anti-IFN-α/-ω antibody comprises a heavy chain variable region (VH) having the amino acid sequence of SEQ ID NO: 28 and a light chain variable region (VL) having the amino acid sequences of SEQ ID NO: 29.

Embodiment 1c is the method of embodiment 1, wherein the anti-IFN-α/-ω antibody is IFWM3405, IFWM3442, IFWM3525, IFWM3423, IFWM3444 or IFWM3421 described in U.S. Pat. No. 10,208,113, the content of which is herein incorporated by reference in its entirety.

Embodiment 1d is the method of any one of embodiments 1 to 1c, wherein the anti-IFN-α/-ω antibody is a fully human antibody.

Embodiment 2 is the method of any of embodiments 1-1d, wherein the pharmaceutical composition is administered intravenously.

Embodiment 2a is the method of embodiment 2, wherein the total dosage of the anti-IFN-α/-ω antibody administered per administration is 0.1 mg/kg, 0.3 mg/kg, 1 mg/kg, 3 mg/kg, 10 mg/kg, 15 mg/kg or 20 mg/kg body weight of the subject, or any dosage in between.

Embodiment 2b is the method of embodiment 2 or 2, wherein the pharmaceutical composition is administered to the human subject intravenously for no less than 30 minutes.

Embodiment 3 is the method of any of embodiments 1-1d, wherein the pharmaceutical composition is administered subcutaneously.

Embodiment 3a is the method of embodiment 3, wherein the total dosage of the anti-IFN-α/-ω antibody administered per administration is 0.1 mg/kg, 0.3 mg/kg, 0.5 mg/kg, 0.7 mg/kg, 1 mg/kg, 1.2 mg/kg, 1.5 mg/kg, 1.7 mg/kg, 2 mg/kg, 2.3 mg/kg or 2.5 mg/kg body weight of the subject, or any dosage in between.

Embodiment 4 is the method of any one of embodiments 1-3a, wherein the administration of the pharmaceutical composition achieves, in the plasma of the subject, at least one parameter selected from: (i) an area under the concentration time curve (AUC)₍₀₋ₜ₎ of about 50 μg.day/mL to about 7000 μg.day/mL, and (ii) a maximum concentration observed (Cₘₐₓ) of about 5 μg/mL to about 500 μg/mL.

Embodiment 4a is the method of embodiment 4, wherein the administration of the pharmaceutical composition achieves, in the plasma of the subject, an area under the concentration time curve (AUC)₍₀₋ₜ₎ of about 50 μg.day/mL, 100 μg.day/mL, 500 μg.day/mL, 1000 μg.day/mL, 2000 μg.day/mL, 3000 μg.day/mL, 4000 μg.day/mL, 5000 μg.day/mL, 6000 μg.day/mL, 7000 μg.day/mL, or any number in between.

Embodiment 4a is the method of embodiment 4, wherein the human subject is a healthy subject, and the intravenous administration of a single dose of 0.3 mg/kg, 1.0 mg/kg, 3.0 mg/kg, 10 mg/kg or 15 mg/kg of the anti-IFN-α/-ω antibody achieves, in the plasma of the subject, an area under the concentration time curve (AUC)₍₀₋₁₎ of 88.8 (12.6), 287 (31,9), 1143 (245), 3694 (610), or 5944 (1036) μg.day/mL, respectively.

Embodiment 4b is the method of embodiment 4 or 4a, wherein the human subject is a healthy subject, and the intravenous administration of a single dose of 0.3 mg/kg, 1.0 mg/kg, 3.0 mg/kg, 10 mg/kg or 15 mg/kg of the anti-IFN-α/-ω antibody achieves, in the plasma of the subject, a maximum concentration observed (Cₘₐₓ) of 7.14 (0.961), 22.5 (2.21), 76.5 (9.30), 230 (22.4), or 403 (65.7) μg/mL, respectively.

Embodiment 4c is the method of embodiment 4, wherein the human subject is a healthy subject, and the subcutaneous administration of a single dose of 1.0 mg/kg of the anti-IFN-α/-ω antibody achieves, in the plasma of the subject, an area under the concentration time curve (AUC)₍₀₋₁₎ of 226 (69.5) μg.day/mL.

Embodiment 4d is the method of embodiment 4 or 4c, wherein the human subject is a healthy subject, and the subcutaneous administration of a single dose of 1.0 mg/kg of the anti-IFN-α/-ω antibody achieves, in the plasma of the subject, a maximum concentration observed (Cₘₐₓ) of 7.96 (2.48) μg/mL.

Embodiment 5 is the method of any one of embodiments 1-4d, wherein the administration of the anti-IFN-α/-ω antibody does not result in production of antibodies against the anti-IFN-α/-ω antibody in the subject.

Embodiment 6 is the method of any of embodiments 1-5, wherein the human subject is in need of a treatment of an IFN-I mediated disease, such as systemic lupus erythematosus (SLE), type I diabetes, psoriasis, primary Sjögren's disease, systemic sclerosis, rheumatoid arthritis, transplant rejection, dermatomyositis, polymyositis, Aicardi-Goutières syndrome, Sting associated vasculopathy with onset in infancy (SAVI) or chronic atypical neutrophilic dermatosis with lipodystrophy and elevated temperature syndrome (CANDLE).

Embodiment 7 is the method of embodiment 6, wherein the human subject is in need of a treatment of mild to moderate systemic lupus erythematosus (SLE).

Embodiment 8 is the method of any one of embodiments 6-7, wherein the pharmaceutical composition is administered to the human subject intravenously.

Embodiment 8a is the method of embodiment 8, wherein a total dosage of the anti-IFN-α/-ω antibody administered is 10 mg/kg body weight of the subject per administration.

Embodiment 8b is the method of embodiment 8 or 8a, wherein the pharmaceutical composition is administered to the human subject once every two weeks.

Embodiment 9 is the method of any one of embodiments 6-7, wherein the pharmaceutical composition is administered to the human subject subcutaneously.

Embodiment 9a is the embodiment 9, wherein a total dosage of the anti-IFN-α/-ω antibody administered is 1 mg/kg body weight of the subject per administration.

Embodiment 10 is the method of any one of embodiments 7-9a, wherein the administration of the pharmaceutical composition achieves, in the plasma of the subject, at least one parameter selected from: (i) an area under the concentration time curve (AUC)_{(0-14d)} of about 800 μg.day/mL to about 3500 μg.day/mL, and (ii) a maximum concentration observed (Cₘₐₓ) of about 100 μg/mL to about 400 μg/mL.

Embodiment 10a is the method of embodiment 10, wherein the pharmaceutical composition is intravenously administered to the human subject once every two weeks at 10 mg/kg of the anti-IFN-α/-ω antibody per administration, and after the first dose, the administration achieves, in the plasma of the subject, an area under the concentration time curve during a dosing interval (AUC)₍₀₋₁₄ₐ₎ of 1387 (354) μg.day/mL.

Embodiment 10b is the method of embodiment 10 or 10a, wherein the pharmaceutical composition is intravenously administered to the human subject once every two weeks at 10 mg/kg of the anti-IFN-α/-ω antibody per administration, and after the sixth dose, the administration achieves, in the plasma of the subject, an area under the concentration time curve during a dosing interval (AUC)_{(0-14d)} of 2340 (690) μg.day/mL.

Embodiment 10c is the method of any one of embodiments 10 to 10b, wherein the intravenous administration of a first dose of 10 mg/kg of the anti-IFN-α/-ω antibody achieves, in the plasma of the subject, a maximum concentration observed (Cₘₐₓ) of 194 (46.3).

Embodiment 10d is the method of any one of embodiments 10 to 10c, wherein the intravenous administration of a sixth dose of 10 mg/kg of the anti-IFN-α/-ω antibody achieves, in the plasma of the subject, a maximum concentration observed (Cₘₐₓ) of 289 (72.6), wherein each dose of the pharmaceutical composition is administered to the human subject once every two weeks.

Embodiment 11 is the method of any one of embodiments 7-10d, wherein the human subject has a reduction in the Systemic Lupus Erythematosus Responder Index (SRI), preferably a reduction of 4 points, more preferably 5 points, and most preferably 6 points, by day 100 after the administration of the pharmaceutical composition.

Embodiment 12 is the method of any one of embodiments 7-11, wherein the human subject has no new British Isles Lupus Assessment Group (BILAG) A or 2B shifts by day 100 after the administration of the pharmaceutical composition.

Embodiment 13 is the method of any one of embodiments 7-12, wherein the human subject has a reduction in the Systemic Lupus Erythematosus Disease Activity Index 2000 (SLEDAI-2K) from baseline by day 100 after the administration of the pharmaceutical composition.

Embodiment 14 is the method of any one of embodiments 7-13, wherein the human subject has a reduction in the Systemic Lupus Erythematosus 2000 Responder Index-50 (S2K RI-50) from baseline by day 100 after the administration of the pharmaceutical composition.

Embodiment 15 is the method of any one of embodiments 7-14, wherein the human subject has a reduction in the Physician's Global Assessment of Disease Activity (PGA) from baseline by day 100 after the administration of the pharmaceutical composition.

Embodiment 16 is the method of any one of embodiments 6-15, wherein the human subject achieves a steady-state condition of the antibody within 40-50 days after administration.

Embodiment 17 is the method of any one of embodiments 6-16, wherein the administration of the anti-IFN-α/-ω antibody does not result in a treatment emergent adverse event (TEAE) related to a malignancy or anaphylactic or serum sickness-type reaction in the subject.

Embodiment 18 is a method of providing clinically proven safe treatment of an IFN-I mediated disease in a human subject in need thereof, the method comprising subcutaneously or intravenously administering to the subject a pharmaceutical composition comprising an anti-IFN-α/-ω antibody and a pharmaceutically acceptable carrier, wherein a total dosage of the anti-IFN-α/-ω antibody administered is 0.1 mg/kg to 20 mg/kg body weight of the subject per administration.

Embodiment 18a is the method of embodiment 18, wherein the anti-IFN-α/-ω antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 33, 34, and 35, respectively, and the light chain variable region comprising light chain complementarity determining regions (LCDRs) LCDR1, LCDR2 and LCDR3 amino acid sequences of SEQ ID NOs: 30, 31, and 32, respectively.

Embodiment 18b is the method of embodiment 18 or 18a, wherein the anti-IFN-α/-ω antibody comprises a heavy chain variable region (VH) having the amino acid sequence of SEQ ID NO: 28 and a light chain variable region (VL) having the amino acid sequences of SEQ ID NO: 29.

Embodiment 18c is the method of embodiment 18, wherein the anti-IFN-α/-ω antibody is IFWM3405, IFWM3442, IFWM3525, IFWM3423, IFWM3444 or IFWM3421 described in U.S. Pat. No. 10,208,113, the content of which is herein incorporated by reference in its entirety.

Embodiment 18d is the method of any one of embodiments 18 to 18c, wherein the anti-IFN-α/-ω antibody is a fully human antibody.

Embodiment 19 is the method of any of embodiments 18-18d, wherein the IFN-I mediated disease is selected from the group consisting of systemic lupus erythematosus (SLE), type I diabetes, psoriasis, primary Sjögren's disease, systemic sclerosis, rheumatoid arthritis, transplant rejection, dermatomyositis, polymyositis, Aicardi-Goutières syndrome, Sting associated vasculopathy with onset in infancy (SAVI) or chronic atypical neutrophilic dermatosis with lipodystrophy and elevated temperature syndrome (CANDLE).

Embodiment 20 is the method of embodiment 19, wherein the human subject is in need of a treatment of mild to moderate systemic lupus erythematosus (SLE).

Embodiment 21 is the method of any one of embodiments 18-20, wherein the pharmaceutical composition is administered to the human subject intravenously for no less than 30 minutes.

Embodiment 21a is the method of embodiment 21, wherein a total dosage of the anti-IFN-α/-ω antibody administered is 10 mg/kg body weight of the subject per administration.

Embodiment 21b is the method of embodiment 21a, wherein the pharmaceutical composition is administered to the human subject once every two weeks.

Embodiment 22 is the method of any one of embodiments 18-20, wherein the pharmaceutical composition is administered to the human subject subcutaneously.

Embodiment 22a is the embodiment 22, wherein a total dosage of the anti-IFN-α/-ω antibody administered is 1 mg/kg body weight of the subject per administration.

Embodiment 23 is the method of any one of embodiments 18-22a, wherein the administration of the anti-IFN-α/-ω antibody does not result in production of antibodies against the anti-IFN-α/-ω antibody in the subject.

Embodiment 24 is the method of any one of embodiments 18-23, wherein the administration of the pharmaceutical composition achieves, in the plasma of the subject, at least one parameter selected from: (i) an area under the concentration time curve (AUC)₍₀₋ₜ₎ of about 50 μg.day/mL to about 7000 μg.day/mL., and (ii) a maximum concentration observed (Cₘₐₓ) of about 5 μg/mL to about 500 μg/mL.

Embodiment 24a is the method of embodiment 24, wherein the administration of the pharmaceutical composition achieves, in the plasma of the subject, at least one parameter selected from: (i) an area under the concentration time curve (AUC)_{(0-14d)} of about 800 μg.day/mL to about 3500 μg.day/mL, and (ii) a maximum concentration observed (Cₘₐₓ) of about 100 μg/mL to about 400 μg/mL.

Embodiment 24b is the method of embodiment 24a, wherein the pharmaceutical composition is intravenously administered to the human subject once every two weeks at 10 mg/kg of the anti-IFN-α/-ω antibody per administration, and after the first dose, the administration achieves, in the plasma of the subject, an area under the concentration time curve during a dosing interval (AUC)_{(0-14d)} of 1387 (354) μg.day/mL.

Embodiment 24c is the method of embodiment 24a or 24b, wherein the pharmaceutical composition is intravenously administered to the human subject once every two weeks at 10 mg/kg of the anti-IFN-α/-ω antibody per administration, and after the sixth dose, the administration achieves, in the plasma of the subject, an area under the concentration time curve during a dosing interval (AUC)_{(0-14d)} of 2340 (690) μg.day/mL.

Embodiment 24d is the method of any one of embodiments 24a to 24c, wherein the intravenous administration of a first dose of 10 mg/kg of the anti-IFN-α/-ω antibody achieves, in the plasma of the subject, a maximum concentration observed (Cₘₐₓ) of 194 (46.3).

Embodiment 24e is the method of any one of embodiments 2 a to 24d, wherein the intravenous administration of a sixth dose of 10 mg/kg of the anti-IFN-α/-ω antibody achieves, in the plasma of the subject, a maximum concentration observed (Cₘₐₓ) of 289 (72.6), wherein each dose of the pharmaceutical composition is administered to the human subject once every two weeks.

Embodiment 25 is the method of any one of embodiments 18-24e, wherein the human subject has a reduction in the Systemic Lupus Erythematosus Responder Index (SRI), preferably a reduction of 4 points, more preferably 5 points, and most preferably 6 points, by day 100 after the administration of the pharmaceutical composition.

Embodiment 26 is the method of any one of embodiments 18-25, wherein the human subject has no new British Isles Lupus Assessment Group (BILAG) A or 2B shifts by day 100 after the administration of the pharmaceutical composition.

Embodiment 27 is the method of any one of embodiments 18-26, wherein the human subject has a reduction in the Systemic Lupus Erythematosus Disease Activity Index 2000 (SLEDAI-2K) from baseline by day 100 after the administration of the pharmaceutical composition.

Embodiment 28 is the method of any one of embodiments 18-27, wherein the human subject has a reduction in the Systemic Lupus Erythematosus 2000 Responder Index-50 (S2K RI-50) from baseline by day 100 after the administration of the pharmaceutical composition.

Embodiment 29 is the method of any one of embodiments 18-28, wherein the human subject has a reduction in the Physician's Global Assessment of Disease Activity (PGA) from baseline by day 100 after the administration of the pharmaceutical composition.

Embodiment 30 is the method of any one of embodiments 18-29, wherein the human subject achieves a steady-state condition of the antibody within 40-50 days after administration.

Embodiment 31 is the method of any one of embodiments 18-30, wherein the administration of the anti-IFN-α/-ω antibody does not result in a treatment emergent adverse event (TEAE) related to a malignancy or anaphylactic or serum sickness-type reaction in the subject.

Embodiment 32 is the method of any one of embodiments 1-31, further comprising:
a. providing a biological sample from the human subject;
b. assaying gene expression of one or more genes DHX58, EIF2AK2, HERC5, IFI44, IFI44L, IFI6, IRF7, PARP9, PLSCR1 and SAMD9L in a biological sample of the human subject; and
c. identifying the human subject as responsive to treatment of the antibody prior to administering to the subject the pharmaceutical composition comprising the antibody and the pharmaceutically acceptable carrier.

Embodiment 33 is the method of embodiment 32, comprising:
a. providing a biological sample from the human subject;
b. assaying gene expression of genes DHX58, EIF2AK2, HERC5, IFI44, IFI44L, IFI6, IRF7, PARP9, PLSCR1 and SAMD9L in a biological sample of the human subject;
c. determining a combined expression value of the genes DHX58, EIF2AK2, HERC5, IFI44, IFI44L, IFI6, IRF7, PARP9, PLSCR1 and SAMD9L in the biological sample; and
d. identifying the human subject as responsive to treatment of the antibody prior to administering to the subject the pharmaceutical composition comprising the antibody and the pharmaceutically acceptable carrier.

Embodiment 34 is the method of embodiment 33, comprising:
a. providing a biological sample from the human subject;
b. assaying gene expression of genes DHX58, EIF2AK2, HERC5, IFI44, IFI44L, IFI6, IRF7, PARP9, PLSCR1 and SAMD9L in a biological sample of the human subject;
c. determining a combined expression value of the genes DHX58, EIF2AK2, HERC5, IFI44, IFI44L, IFI6, IRF7, PARP9, PLSCR1 and SAMD9L in the biological sample;
d. determining that the human subject has an elevated IFN-I signature when the combined expression value is equal to or higher than a threshold value; and
b. identifying the human subject as responsive to treatment of the antibody prior to administering to the subject the pharmaceutical composition comprising the antibody and the pharmaceutically acceptable carrier.

Embodiment 35 is the method of any one of embodiments 1 to 34, wherein the pharmaceutical composition comprises the anti-IFN-α/-ω antibody at a concentration of 1 mg/mL to 100 mg/mL, and a pharmaceutically acceptable carrier.

Embodiment 35a is the method of embodiment 35, wherein the concentration of the anti-IFN-α/-ω antibody is 1 mg/mL, 10 mg/mL, 20 mg/mL, 30 mg/mL, 40 mg/mL, 50 mg/mL, 60 mg/mL, 70 mg/mL, 80 mg/mL, 90 mg/mL, or 100 mg/mL, or any concentration in between.

Embodiment 35b is the method of embodiment 35 or 35a, wherein the pharmaceutically acceptable carrier comprises one or more carbohydrates, such as lactose, maltose, sucrose, at a concentration of 1% to 10% weight by volume (w/v), such as 5% to 10% (w/v).

Embodiment 35c is the method of embodiment 35b, wherein the pharmaceutically acceptable carrier comprises at least one of lactose, maltose and sucrose, at a concentration of 1% (w/v), 1.5% (w/v), 2% (w/v), 2.5% (w/v), 3% (w/v), 3.5% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), or 10% (w/v), or any concentration in between.

Embodiment 35d is the method of any one of embodiments 35 to 35c, wherein the pharmaceutically acceptable carrier comprises one or more surfactants, such as polysorbate 20, polysorbate 80 at a concentration of 0.01% (w/v) to 0.1% (w/v) or 0.02% (w/v) to 0.08% (w/v).

Embodiment 35e is the method of embodiment 35d, wherein the pharmaceutically acceptable carrier comprises at least one of polysorbate 20 and polysorbate 80 at a concentration of 0.01%, 0.02%, 0.03%, 0.04%, 0.05%, 0.06%, 0.07%, 0.08%, 0.09% or 0.1% (w/v), or any concentration in between.

Embodiment 35f is the method of any one of embodiments 35 to 35e, wherein the pharmaceutically acceptable carrier comprises one or more acids and/or salts, such as lactic acid, sodium lactate, acetic acid, sodium acetate at a concentration of 10 mM to 20 mM based on the amount of lactate or acetate, respectively.

Embodiment 35g is the method of embodiment 35f, wherein the pharmaceutically acceptable carrier comprises at least one of sodium lactate and sodium acetate at a concentration of 10 mM, 11 mM, 12 mM, 13 mM, 14 mM, 15 mM, 16 mM, 17 mM, 18 mM, 19 mM, 20 mM, or any concentration in between.

Embodiment 35h is the method of any one of embodiments 35 to 35g, wherein the pharmaceutical composition has a pH of 5.0-6.0, such as a pH of 5.0, 5.1, 5.2, 5.3, 5.4, 5.5., 5.6, 5.7, 5.8, 5.9, 6.0, or any value in between.

Embodiment 36 is the method of any one of embodiments 1 to 34, wherein the pharmaceutical comprises 20 mg/mL to 80 mg/mL of the anti-IFN-α/-ω antibody and a pharmaceutically acceptable carrier.

Embodiment 36a is the method of embodiment 36, wherein the pharmaceutically acceptable carrier comprises 1 mM to 20 mM acetate.

Embodiment 36b is the method of embodiment 36, wherein the pharmaceutically acceptable carrier comprises 1 mM to 20 mM lactate.

Embodiment 36c is the method of any one of embodiments 36 to 36b, wherein the pharmaceutically acceptable carrier comprises 5% to 10% (w/v) lactose.

Embodiment 36d is the method of any one of embodiments 36 to 36b, wherein the pharmaceutically acceptable carrier comprises 5% to 10% (w/v) maltose.

Embodiment 36e is the method of any one of embodiments 36 to 36b, wherein the pharmaceutically acceptable carrier comprises 5% to 10% (w/v) sucrose.

Embodiment 36f is the method of any one of embodiments 36 to 36e, wherein the pharmaceutically acceptable carrier comprises 0.01% to 0.10% (w/v) polysorbate 20 (PS20).

Embodiment 36g is the method of any one of embodiments 36 to 36e, wherein the pharmaceutically acceptable carrier comprises 0.01% to 0.10% (w/v) polysorbate 80 (PS80).

Embodiment 36h is the method of any one of embodiments 36 to 36g, wherein the pharmaceutical composition has a pH of 5.0-6.0, such as a pH of 5.0, 5.1, 5.2, 5.3, 5.4, 5.5., 5.6, 5.7, 5.8, 5.9, 6.0, or any value in between.

Embodiment 37 is the method of any one of embodiments 1 to 34, wherein the pharmaceutical comprises 30 mg/mL of the anti-IFN-α/-ω antibody, 5 mM lactate, 5% (w/v) lactose, and 0.02% (w/v) polysorbate 80 (PS80), at pH 5.5.

Embodiment 38 is the method of any one of embodiments 1 to 34, wherein the pharmaceutical comprises 40 mg/mL of the anti-IFN-α/-ω antibody, 8 mM acetate, 8% (w/v) maltose, and 0.06% (w/v) polysorbate 20 (PS20), at pH 5.0.

Embodiment 39 is the method of any one of embodiments 1 to 34, wherein the pharmaceutical composition comprises 50 mg/mL of the anti-IFN-α/-ω antibody, 13 mM acetate, 8.0% (w/v) sucrose, and 0.04% (w/v) polysorbate 20 (PS20), at pH 5.2.

Embodiment 40 is the method of any one of embodiments 1 to 34, wherein the pharmaceutical comprises 60 mg/mL of the anti-IFN-α/-ω antibody, 10 mM acetate, 8% (w/v) lactose, and 0.06% (w/v) polysorbate 20 (PS20), at pH 5.4.

Embodiment 41 is the method of any one of embodiments 1 to 34, wherein the pharmaceutical comprises 70 mg/mL of the anti-IFN-α/-ω antibody, 15 mM lactate, 10% (w/v) maltose, and 0.06% (w/v) polysorbate 80 (PS80), at pH 5.0.

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### EXAMPLES

### Example 1: A Phase 1, Randomized, Double-blind, Placebo-controlled, Single Ascending Dose Study in Healthy Subjects and Multiple Dose Study of ANTIBODY A in Subjects With Mild to Moderate Systemic Lupus Erythematosus

This clinical study was the first administration of a fully human anti-IFN-α/-ω antibody (“ANTIBODY A”) in human. The antibody A has a heavy chain variable region (VH) amino acid sequence of SEQ ID NO: 28 and a light chain variable region (VL) amino acid sequences of SEQ ID NO: 29 and is an IgG1κ. The purpose of this study was to assess the safety and tolerability of ANTIBODY A through single ascending intravenous (IV) dose administration and a single subcutaneous (SC) dose in healthy subjects (Part A) and multiple IV dose administrations in subjects with mild to moderate systemic lupus erythematosus (SLE) (Part B).

### Objectives

### Primary Objectives

1) Assess the safety and tolerability of ANTIBODY A following single ascending IV dose administration in healthy subjects and a single SC dose of 1 mg/kg in healthy subjects (Part A);
2) Assess the safety and tolerability of ANTIBODY A following multiple IV dose administrations in subjects with mild to moderate SLE (Part B).

### Secondary Objectives

1) Assess the PK and immunogenicity of ANTIBODY A following single ascending IV dose administration in healthy subjects and a single SC dose of 1 mg/kg in healthy subjects (Part A), and following multiple IV dose administrations in subjects with mild to moderate SLE (Part B);
2) Evaluate pharmacodynamic (PD) effects and clinical responses following a single IV or SC dose of ANTIBODY A in healthy subjects (Part A), and evaluate PD and clinical response following multiple IV doses of ANTIBODY A in subjects with mild to moderate SLE (Part B).

### Exploratory Objectives

1) Evaluate biomarkers following a single IV or SC dose of ANTIBODY A in healthy subjects (Part A), and following multiple IV doses of ANTIBODY A in subjects with mild to moderate SLE (Part B);
2) Evaluate the level of dysregulation of IFN signaling and how this dysregulation correlates with changes in other biomarkers and clinical response measures to administration of study agent;
3) Explore the variability of IFN signatures across different racial/ethnic populations and its potential impact on clinical response associated with exposure to study agent;
4) Explore PK/PD relationships of ANTIBODY A through analysis of biomarkers, PD markers, and clinical response.

### Methods and Subjects

### Overview of Study Design

Part A of the study assessed the safety, tolerability, PK, and immunogenicity of a single administration of ANTIBODY A or placebo in healthy subjects with no confounding significant medical problems or chronic medications, where frequent PK sampling was obtained, and biomarkers and PD were also assessed. Single ascending IV doses of 0.3 mg/kg, 1.0 mg/kg, 3.0 mg/kg, 10.0 mg/kg, or 15.0 mg/kg of ANTIBODY A or placebo were administered to sequential cohorts of healthy subjects as an IV infusion of at least 30 minutes. An additional cohort received a single 1 mg/kg SC administration of ANTIBODY A.

Part B of the study, in subjects with mild to moderate SLE, explored clinical response, safety, and tolerability as well as assessed impact on biomarkers such as the target engagement (total and free IFN), IFN-related RNA expression signature, and associated proteins over time. Additional investigation of incremental effect on biomarker readouts were performed to assess variability in the ability to attenuate IFN-mediated signaling across different racial/ethnic populations. If appropriate, subgroup analyses of clinical response and safety parameters were to be performed to evaluate variability and explore the effects of treatment across different subpopulations. In Part B, 6 doses of 10 mg/kg ANTIBODY A or placebo were administered every 2 weeks as an IV infusion of at least 30 minutes. Randomization was stratified by racial/ethnic subpopulation (Asian/non-Asian) and elevated level of serologic disease activity (present/absent).

The duration of subject participation was approximately 13 weeks for healthy subjects and 22 weeks for SLE subjects, including a screening visit up to 28 days prior to study agent administration. The healthy subjects had a 6-day/5-night inpatient period. All subjects received study agent on day 1 and SLE subjects received additional doses on Days 15, 29, 43, 57, and 71.

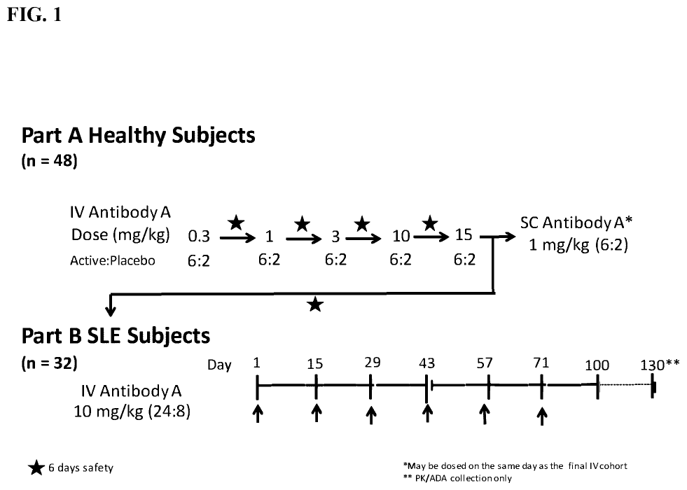

A diagram of the study design is provided in FIG. 1.

### Study Agent

ANTIBODY A is supplied as a frozen, preservative-free, final liquid-in-vial (LIV) presentation suitable for IV and SC administration.

### Subjects

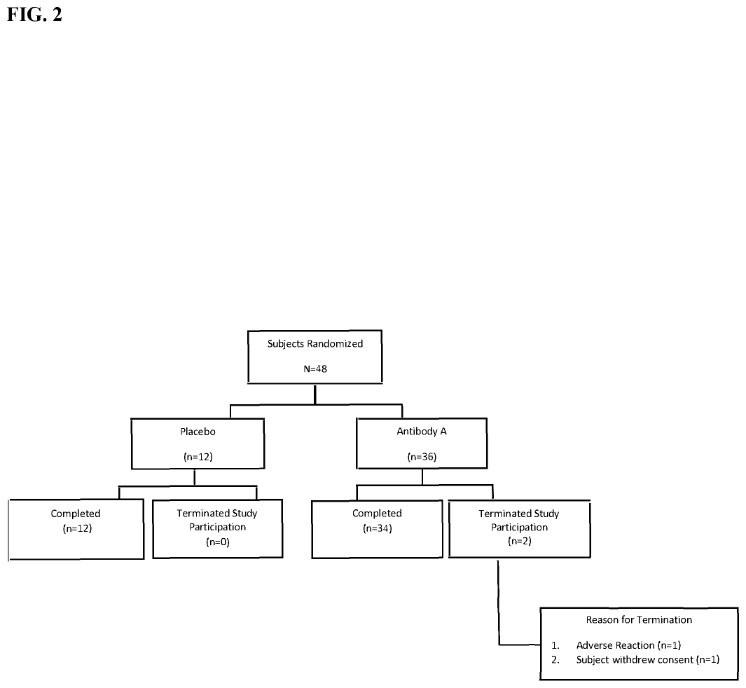

Part A: A total of 48 healthy adult subjects (40 male; 8 female) without no confounding significant medical problems or chronic medications were enrolled. 30 subjects received single ascending IV doses (0.3 to 15.0 mg/kg) of ANTIBODY A, 6 subjects received SC doses (1.0 mg/kg), and 12 subjects received placebo. Two subjects from the ANTIBODY A group did not complete the study. FIG. 2 represents the disposition of subjects in Part A.
Part B: Subjects eligible for enrollment had to meet the following key criteria:
3) Must meet Systemic Lupus International Collaborating Clinics (SLICC) criteria for diagnosis of lupus with at least 1 Systemic Lupus Erythematosus Disease Activity Index 2000 (SLEDAI-2K)-defined non-serologic clinical activity within 3 months prior to first dose of study agent.
4) In addition to meeting SLICC criteria, must be serologically defined as positive within 2 months prior to, or at screening:
e) a positive anti-nuclear antibody (ANA) titer of ≥1:80, or
f) a positive anti-double stranded deoxyribonucleic acid test (note: if Anti-dsDNA is done
by enzyme-linked immunosorbent assay, a test 2× upper limit of normal is required to be considered unequivocally positive) or
g) a positive anti-Smith antibody and/or positive anti-ribonucleoprotein (RNP) antibody and/or anti-Ro antibody, and in addition to at least one of the above:
h) a positive lupus IFN profile score during screening (prior to randomization).

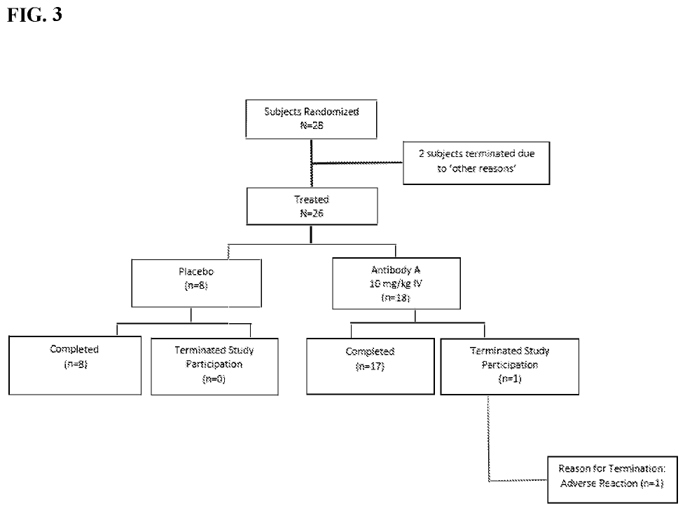

A total of 28 subjects (27 females: 1 male) with mild to moderate SLE were randomized in Part B (20 subjects: 10 mg/kg IV dose; 8 subjects: placebo). Three subjects from the ANTIBODY A group did not complete the study. FIG. 3 resents the disposition of subjects in Part B.

### Evaluations

### Safety Evaluations

The safety and tolerability of ANTIBODY A including physical examinations, infusion reactions, injection site reactions, clinical laboratory tests, vital signs, concomitant medications, and treatment-emergent adverse events (TEAEs) were summarized by treatment groups. The safety and tolerability of ANTIBODY A including physical examinations, infusion reactions, injection site.

### Pharmacokinetic and Immunogenicity

For all subjects participating in the study, serum samples were used to evaluate the PK, as well as the immunogenicity of ANTIBODY A. Serum samples were collected at specified times relative to the start of the infusion/injection as listed in FIG. 1.

Serum samples were analyzed to determine concentrations of ANTIBODY A using a validated, immunoassay method with a lower limit of quantification (LLOQ) of 0.06 μg/mL. The detection and characterization of antibodies to ANTIBODY A was performed using a validated assay method.

### Pharmacodynamic Evaluation

Biopsy of skin lesions from subjects who provided (optional) biopsy consent, pre-and post-treatment biopsies of lesioned skin (2 pre-and 2 post-treatment; 4 mm punch) were taken. Biopsy specimens were to be assessed by mRNA expression and specific immunohistochemistry staining subject to adequacy of the specimen.

### Clinical Response Evaluations

Response evaluations and patient reported quality of life measures were to include SLEDAI-2K/SLEDAI-2K Responder Index (S2K RI-50), British Isles Lupus Assessment Group (BILAG), Cutaneous Lupus Erythematosus Disease Area and Severity Index (CLASI), Physician's Global Assessment (PGA) of Disease Activity, Short-form-36 questionnaire, EuroQol-5 dimensions-5 levels (EQ-5D-5L) Patient Diary, Joint Assessment, and Photography of SLE subject skin lesions for subjects who provided consent to photography.

### Glycoform Analysis

In Part A, subjects receiving 15 mg/kg had serum samples collected and stored. The samples could be analyzed in the future to determine the potential change in ANTIBODY A glycoform levels.

### Biomarkers

Biomarkers were to be analyzed as part of the exploratory analyses for subjects in Parts A and B. This included serum levels of IFNs as well as molecular pathway profiling. These biomarkers included, but were not limited to, inflammatory markers, RNA, cell surface markers, autoantibodies, T-cell and B-cell repertoire, target-specific markers, and other categories of biomarkers potentially involved in the development and the progression of SLE.

### Pharmacogenomic Evaluations

Samples were to be analyzed for the variability of RNA, DNA, protein expression, and changes in IFN signatures. Additional analyses were to be conducted if it was hypothesized that this can help resolve issues with the clinical data.

### Statistical Methods

Clinical response analyses in Part B were to include summaries of SLE evaluations, including organ domain-specific changes.

Pharmacokinetics: Individual subject serum concentration-time data of ANTIBODY A were analyzed by non-compartmental analysis (NCA) and summarized by treatment group and planned sampling time point. PK parameters were summarized by treatment group and PK data were displayed graphically. Plots of the mean serum concentrations of ANTIBODY A over time were provided. Descriptive statistics were calculated for all individual derived PK parameters including exposure information of ANTIBODY A.

Immunogenicity: The status of antibodies to ANTIBODY A were summarized for all subjects who received a dose of ANTIBODY A and had appropriate samples for detection of antibodies to ANTIBODY A. Subjects with baseline positive samples and without increased titer post treatment were not considered anti-drug antibody (ADA) positive.

All TEAEs were summarized by treatment group and MedDRA versions 18.1 (Part A) and 21.0 (Part B), SOC and PTs for each group of healthy and SLE subjects.

### Safety Results

The safety analysis sets included 48 subjects in Part A and 26 subjects in Part B. Overall, 39 (81.3%) subjects in Part A and 20 (76.9%) subjects in Part B reported ≥1 TEAEs during the course of study. The most common TEAE was reported in the system organ class of Infections and infestations for both the parts of the study.

In Part A, 1 subject treated with ANTIBODY A 0.3 mg/kg IV experienced a non-serious TEAE of myringitis bullosa of the right ear. The event was reported as resolved. No infusion reactions were reported and no local injection site reactivity was attributed to the study agent. No Serious TAEs were reported in Part A.

In Part B, serious TEAEs were reported by 2 (7.7%) subjects from the ANTIBODY A cohort: herpes zoster (HZ) by 2 (7.7%) subjects and premature labor by 1 (3.8%) subject. There were no serious TEAEs reported in the placebo cohort. One subject treated with ANTIBODY A 10.0 mg/kg IV discontinued the study due to a non-serious TEAE of groin pain (lymphadenopathy) which was considered to be possibly related to ANTIBODY A. No infusion reactions were reported and no local injection site reactivity was attributed to the study agents. Additionally, no TEAEs related to malignancy and no anaphylactic or serum sickness-type reactions were reported.

Overall, ANTIBODY A was well-tolerated by the normal healthy volunteers and SLE subjects. There were no deaths reported in the study.

In addition, no clinically significant changes from baseline were observed for laboratory parameters, vital signs, physical exam, or ECG findings.

### Pharmacokinetics Results

### Part A

Separate cohorts of healthy subjects received single ascending IV doses over a dose range of 0.3 to 15 mg/kg ANTIBODY A or placebo. In addition, one cohort of healthy subjects received a single SC dose of 1 mg/kg ANTIBODY A or placebo.

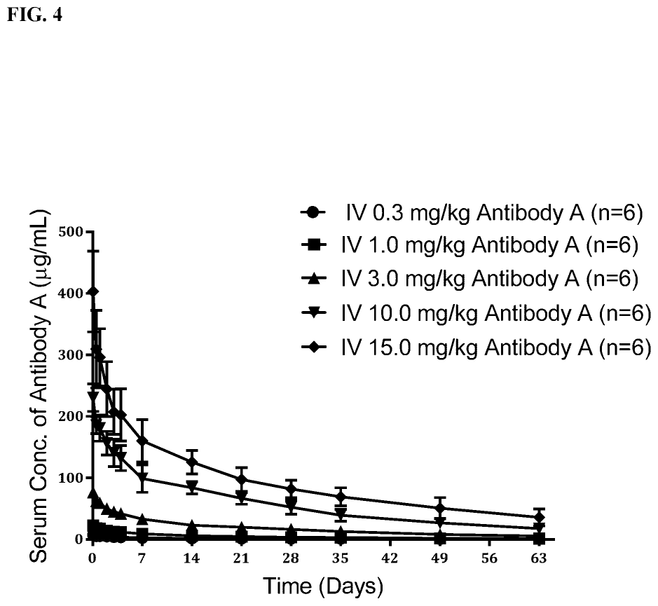

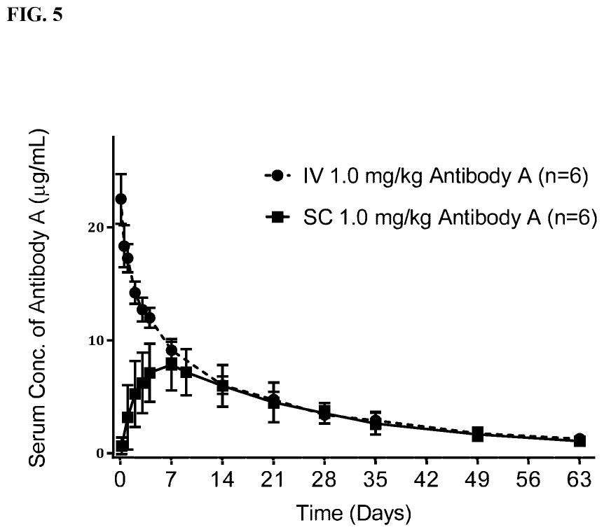

The linear mean serum concentration-time profiles of ANTIBODY A of Cohorts 1 to 5 (IV infusion) are presented in FIG. 4. The linear mean serum concentration-time profile of ANTIBODY A of Cohort 2 (1.0 mg/kg, IV infusion) and Cohort 6 (1.0 mg/kg, SC injection) is presented in FIG. 5.

A summary list of key PK parameters of ANTIBODY A for Cohorts 1 to 5 is presented in Table 11 below. Overall, in Part A, the maximum observed serum concentration of ANTIBODY A (Cmax) and area under the curve (AUC) demonstrated an approximately dose-dependent and dose-proportional increase in the dose range of 0.3 mg/kg to 15 mg/kg, after single IV infusion in healthy subjects. Mean total systemic clearance after IV administration (CL) and volume of distribution at terminal phase (Vz) after a single IV infusion ranged between 2.28 and 3.09 mL/day/kg and between 67.2 and 102 mL/kg, respectively.

TABLE 11
Pharmacokinetic Results of ANTIBODY A After a Single Intravenous Infusion
of ANTIBODY A at Doses Ranging from 0.3 to 15 mg/kg in Healthy Subjects
(Cohorts 1-5)

| Pharmacokinetics of | Single IV infusion of ANTIBODY A | | | | | | | | | |
|---|---|---|---|---|---|---|---|---|---|---|
| ANTIBODY A | 0.3 mg/kg | | 1.0 mg/kg | | 3.0 mg/kg | | 10 mg/kg | | 15 mg/kg | |
| (mean [SD]) | (Cohort 1) | | (Cohort 2) | | (Cohort 3) | | (Cohort 4) | | (Cohort 5) | |
| n | 6^{a} | | 6 | | 6^{b} | | 6^{b} | | 6^{c} | |
| Cₘₐₓ (μg/mL) | 7.14 | (0.961) | 22.5 | (2.21) | 76.5 | (9.30) | 230 | (22.4) | 403 | (65.7) |
| AUC₀₋ₜ (μg · day/mL) | 88.8 | (12.6) | 287 | (31.9) | 1143 | (245) | 3694 | (610) | 5944 | (1036) |
| AUC_{0-inf} (μg · day/mL) | 105 | (18.6) | 331 | (54.7) | 1225 | (334) | 3999 | (621) | 6780 | (1259) |
| T_{1/2} (day) | 24.6 | (3.8) | 22.4 | (5.1) | 20.7 | (6.4) | 22.9 | (4.3) | 24.1 | (2.4) |
| CL (mL/day/kg) | 2.95 | (0.675) | 3.09 | (0.519) | 2.59 | (0.645) | 2.55 | (0.398) | 2.28 | (0.446) |
| V_{Z} (mL/kg) | 102 | (4.54) | 97.2 | (13.1) | 67.2 | (7.98) | 78.8 | (9.04) | 78.1 | (8.57) |
| C_{max, dose normalized} (μg/mL) | 23.8 | (3.20) | 22.5 | (2.21) | 25.5 | (3.10) | 23.0 | (2.24) | 26.9 | (4.38) |
| AUC_{0-t, dose normalized} | 296 | (41.9) | 287 | (31.9) | 381 | (81.6) | 369 | (61.0) | 396 | (69.1) |
| (μg · day/mL) | | | | | | | | | | |
| AUC_{0-inf, dose normalized} | 350 | (61.9) | 331 | (54.7) | 408 | (111) | 400 | (62.1) | 452 | (83.9) |

(μg · day/mL)

^{a}n = 5 for AUC₀₋ₜ, AUC_{0-inf}, CL, V_{Z}, AUC₀₋ₜ, dose normalized, and AUC_{0-inf}, dose normalized
^{b}n = 5 for AUC_{0-inf}, CL, V_{Z}, and AUC_{0-inf}, dose normalized
^{c}n = 5 for AUC_{0-inf}, T_{1/2}, CL, V_{Z}, and AUC_{0-inf}, dose normalized

A summary list of key PK parameters of ANTIBODY A for Cohorts 2 and 6 is presented in Table 2 below. After a single SC injection, mean Cmax (7.96 μg/mL) was reached after 7 days (Tₘₐₓ). Mean total systemic clearance after IV administration/(CL/F) and Vz/F were 3.96 mL/day/kg and 119 mL/kg, respectively. Terminal half-life (T1/2) was similar after IV infusion and SC injection. Mean T1/2 after a single IV infusion ranged from 20.7 to 24.6 days, and mean T1/2 after a SC injection was 22.6 days. The bioavailability of ANTIBODY A administered as a SC injection, based on the comparison with an IV infusion at the same dose, averaged 78.7% and 81.9% for FAUCO-t and FAUCO-inf, respectively.

TABLE 2
Pharmacokinetic Results of ANTIBODY A After a Single Intravenous
Infusion of ANTIBODY A 1.0 mg/kg (Cohort 2)
or After a Single Subcutaneous Injection of
ANTIBODY A 1.0 mg/kg (Cohort 6) in Healthy Subjects

| Pharmacokinetics | 1.0 mg/kg IV | 1.0 mg/kg |
|---|---|---|
| of ANTIBODY A | infusion of | SC injection of |
| (mean [SD], | ANTIBODY A | ANTIBODY A |
| Tₘₐₓ: median [range]) | (Cohort 2) | (Cohort 6) |
| n | 6 | 5^{a} |
| Cₘₐₓ (μg/mL) | 22.5 (2.21) | 7.96 (2.48) |
| Tₘₐₓ (day) | — | 6.96 (2.00-7.00) |
| AUC₀₋ₜ (μg · day/mL) | 287 (31.9) | 226 (69.5) |
| AUC_{0-inf} (μg · day/mL) | 331 (54.7) | 271 (87.3) |
| T_{1/2} (day) | 22.4 (5.1) | 22.6 (4.1) |
| CL(/F) (mL/day/kg) | 3.09 (0.519) | 3.96 (1.14) |
| V_{z}(/F) (mL/kg) | 97.2 (13.1) | 119 (37.0) |
| F_{AUC0-t} (%) | — | 78.7 (24.2) |
| F_{AUC0-inf} (%) | — | 81.9 (26.4) |

^{a}n = 4 for AUC_{0-inf}, CL/F, V_{z}/F, and F_{AUC0-inf}

### Part B

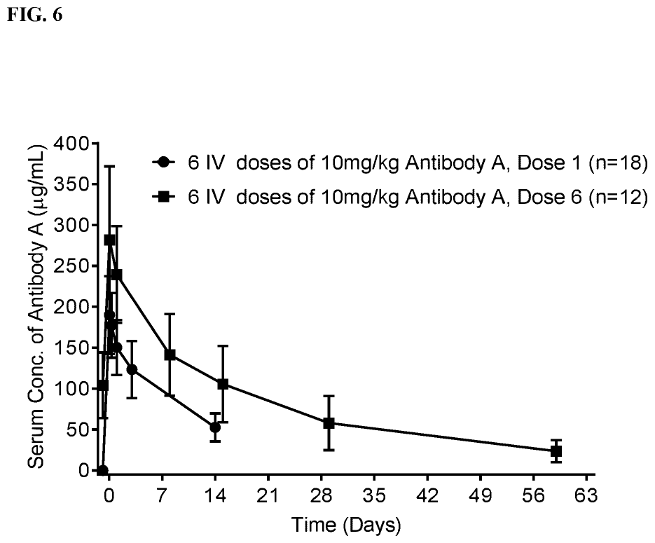

In Part B of this study, 28 subjects with mild to moderate SLE were randomized to receive 6 doses of 10 mg/kg ANTIBODY A or placebo administered every 2 weeks as an IV infusion of at least 30 minutes. The linear mean serum concentration-time profiles of ANTIBODY A of Dose 1 and Dose 6 are presented in FIG. 6.

Following 6 doses of IV infusions of ANTIBODY A at 10 mg/kg every 2 weeks, the shape of the PK profiles of Dose 1 and Dose 6 were generally comparable up till 14 days postdose, i.e., 1 dosing interval. The PK profiles after Dose 1 and Dose 6 were similar to the PK profiles observed in healthy volunteers, with a bi-phasic disposition. Peak serum concentrations were approximately 1.5 times higher after Dose 6 compared to Dose 1. The exposure of ANTIBODY A during a dosing interval (areas under the curve for 0 to 14 days [AUC0-14d]) was higher after Dose 6 as compared to Dose 1.

A summary list of key PK parameters of ANTIBODY A is presented in Table 3 below. Based on the pre-dose plasma concentration (C_{predose}) concentrations of Dose 4, 5, and 6, steady-state conditions of ANTIBODY A appeared to be achieved within 43 days of treatment (Dose 4) with ANTIBODY A. Mean accumulation based on AUC was 1.86-fold. Mean accumulation based on Cmax was 1.62-fold after Dose 6 as compared to Dose 1. Mean values for CL and volume of distribution (Vₛₛ) after the sixth dose were 4.73 mL/day/kg and 92.6 mL/kg, respectively. Mean T1/2 after the 6th dose was 14.8 days.

Although no formal comparison has been performed, the exposure in subjects with mild to moderate SLE seems slightly lower when compared to healthy subjects at an IV dose of 10 mg/kg.

TABLE 3
Pharmacokinetic Results of ANTIBODY A After Multiple
Intravenous Infusions of 10 mg/kg ANTIBODY A in Subjects
with Mild to Moderate Systemic Lupus Erythematosus

| Pharmacokinetics of ANTIBODY A | 10 mg/kg IV infusion |
|---|---|
| (mean [SD]) | of ANTIBODY A |
| Dose 1 | |
| n | 18^{a} |
| Cₘₐₓ (μg/mL) | 194 (46.3) |
| AUC_{0-14d} (μg · day/mL) | 1387 (354) |
| Dose 4 | |
| n | 17 |
| C_{predose} (μg/mL) | 107 (59.9) |
| Dose 5 | |
| n | 16 |
| C_{predose} (μg/mL) | 101 (38.9) |
| Dose 6 | |
| n | 16^{b} |
| C_{predose} (μg/mL) | 104 (40.2) |
| Cₘₐₓ (μg/mL) | 289 (72.6) |
| AUC_{0-14d} (μg · day/mL) | 2340 (690) |
| T_{1/2} (day) | 14.8 (4.4) |
| CL (mL/day/kg) | 4.73 (1.78) |
| Vₛₛ (mL/kg) | 92.6 (23.9) |
| AR_{CMAX} (%) | 162.34 (74.50) |
| AR_{AUC} (%) | 186.12 (115.15) |

^{a}n = 17 for AUC_{0-14d}
^{b}n = 17 for C_{predose}

### Immunogenicity Results (Part A and Part B)

No subject developed antibody to ANTIBODY A following single administration of ANTIBODY A IV between 0.3 to 15 mg/kg or SC at 1 mg/kg in healthy subjects or multiple administrations of ANTIBODY A IV at 10 mg/kg in subjects with mild to moderate SLE. Therefore, no ADA incidence was calculated and reported.

### Clinical Response Results from Part B

### SLE Responder Index (SRI)-4, 5, and 6

The number of subjects with SRI-4, SRI-5 or SRI-6 response at day 100 is presented in Table 4. Data from the overall SRI response at day 100 indicates that subjects from the ANTIBODY A treatment group demonstrated numerically greater response rate than those in the placebo cohort (31.3% versus 0%, respectively).

TABLE 4
Number of Subjects with SRI-4, SRI-5 or
SRI-6 Response at day 100 in Part B; Full Analysis Set

| | | ANTIBODY |
|---|---|---|
| | | A 10 mg/kg |
| | Placebo | IV |
| Analysis Set: Full Analysis Set | 8 | 18 |
| Subjects evaluable for SRI response at day 100 | 8 | 16 |
| Number of subjects with SRI-4 response (%) | 0 | 5 (31.3%) |
| Number of subjects with SRI-5 response (%) | 0 | 2 (12.5%) |
| Number of subjects with SRI-6 response (%) | 0 | 2 (12.5%) |

Note:
SRI-4 response defined as ≥4 point reduction in SLEDAI-2K Total score, no new domain scores in either BILAG A or BILAG B and no worsening (<1 cm increase) from baseline in the Physician's Global Assessment of Disease Activity (PGA). SRI-5 and SRI-6 are defined similarly however a 5-point or 6-point reduction in SLEDAI-2K, respectively, is required.

### BILAG

No new BILAG A or 2B shifts were noted, as shown in Table 5 below.

TABLE 5
Summary of Time to First SLEDAI, Severe SLEDAI
or BILAG Flare from day 1 through day 100 in Part B

| | | ANTIBODY |
|---|---|---|
| | | A 10 mg/kg |
| | Placebo | IV |
| Analysis Set: Full Analysis Set | 8 | 18 |
| Subjects with SLEDAI Flare from | 1 (12.5%) | 3 (16.7%) |
| day 1 to day 100 | | |
| Time to First SLEDAI Flare | | |
| Mean (SD) | 18.0 (—) | 25.0 (8.66) |
| Median | 18.0 | 30.0 |
| Range | (18; 18) | (15; 30) |
| Subjects with Severe SLEDAI Flare | 0 | 0 |
| from day 1 to day 100 | | |
| Time to First Severe SLEDAI Flare | | |
| Mean (SD) | — | — |
| Median | — | — |
| Range | — | — |
| Subjects with BILAG Flare from day | 0 | 0 |
| 1 to day 100 | | |
| Time to First BILAG Flare | | |
| Mean (SD) | — | — |
| Median | — | — |
| Range | — | — |
| Subjects with SLEDAI, Severe SLEDAI | 1 (12.5%) | 3 (16.7%) |
| or BILAG Flare from day 1 to day 100 | | |
| Time to First SLEDAI, Severe SLEDAI | | |
| or BILAG Flare | | |
| Mean (SD) | 18.0 (—) | 25.0 (8.66) |
| Median | 18.0 | 30.0 |
| Range | (18; 18) | (15; 30) |

Note:
SLEDAI flare defined as ≥4 point increase in SLEDAI-2K score. Severe SLEDAI flare defined as ≥7 point increase in SLEDAI-2K score. BILAG flare defined as at least 1 new BILAG A or 2 new BILAG B scores (from scores < B). Time to flare defined as the time (in days) post baseline when the first flare occurs.

### SLEDAI-2K

A summary of change and percent change from baseline in SLEDAI-2K over time are presented in Table 6. Overall, data from the ANTIBODY A cohort demonstrated numerically greater reduction that that from the placebo cohort: mean (SD) percentage change from baseline at day 100 for SLEDAI-2K: −23.74 (27.061) versus −8.93 (13.139), respectively.

TABLE 6
Summary of Change and Percent Change from Baseline in
SLEDAI-2K over Time in Part B

| | | ANTIBODY |
|---|---|---|
| | | A 10 mg/kg |
| | Placebo | IV |
| Analysis Set: Full Analysis Set | 8 | 18 |
| SLEDAI-2K (0-105) | | |
| Baseline Mean (SD) | 9.5 (3.16) | 8.9 (3.64) |
| Baseline Median | 10.0 | 8.5 |
| Subjects evaluable at day 15 | 6 | 15 |
| Baseline Mean (SD) | 9.7 (1.97) | 9.1 (3.91) |
| Baseline Median | 10.0 | 9.0 |
| Change from baseline | | |
| Mean (SD) | −0.3 (3.88) | −0.7 (2.97) |
| Median | 0.0 | 0.0 |
| Range | (−6; 6) | (−8; 6) |
| IQ range | (−2.0; 0.0) | (−2.0; 0.0) |
| Percent change from baseline | | |
| Mean (SD) | 5.00 (50.498) | −1.71 (30.468) |
| Median | 0.00 | 0.00 |
| Range | (−50.0; 100.0) | (−40.0; 75.0) |
| IQ range | (−20.00; 0.00) | (−25.00; 0.00) |
| Subjects evaluable at Day 29 | 7 | 16 |
| Baseline Mean (SD) | 10.3 (2.43) | 9.2 (3.78) |
| Baseline Median | 10.0 | 9.5 |
| Change from baseline | | |
| Mean (SD) | −1.3 (2.63) | 0.1 (3.54) |
| Median | 0.0 | 0.0 |
| Range | (−6; 2) | (−8; 6) |
| IQ range | (−3.0; 0.0) | (−2.0; 2.0) |
| Percent change from baseline | | |
| Mean (SD) | −8.30 (25.770) | 8.13 (37.991) |
| Median | 0.00 | 0.00 |
| Range | (−50.0; 33.3) | (−50.0; 75.0) |
| IQ range | (−21.40; 0.00) | (−16.70; 35.00) |
| Subjects evaluable at Day 57 | 6 | 16 |
| Baseline Mean (SD) | 9.7 (1.97) | 9.2 (3.78) |
| Baseline Median | 10.0 | 9.5 |
| Change from baseline | | |
| Mean (SD) | 0.7 (3.27) | −1.9 (2.74) |
| Median | 0.0 | −1.0 |
| Range | (−4; 6) | (−8; 2) |
| IQ range | (0.0; 2.0) | (−4.0; 0.0) |
| Percent change from baseline | | |
| Mean (SD) | 14.45 (45.290) | −16.39 (29.462) |
| Median | 0.00 | −8.35 |
| Range | (−33.3; 100.0) | (−75.0; 50.0) |
| IQ range | (0.00; 20.00) | (−40.00; 0.00) |
| Subjects evaluable at Day 71 | 8 | 16 |
| Baseline Mean (SD) | 9.5 (3.16) | 9.2 (3.78) |
| Baseline Median | 10.0 | 9.5 |
| Change from baseline | | |
| Mean (SD) | −1.0 (2.98) | −2.8 (4.06) |
| Median | −0.5 | −2.0 |
| Range | (−6; 3) | (−16; 0) |
| IQ range | (−3.0; 1.0) | (−3.0; 0.0) |
| Percent change from baseline | | |
| Mean (SD) | −4.83 (30.547) | −25.76 (27.567) |
| Median | −5.00 | −19.45 |
| Range | (−50.0; 50.0) | (−80.0; 0.0) |
| IQ range | (−24.30; 10.00) | (−41.65; 0.00) |
| Subjects evaluable at day 100 | 8 | 16 |
| Baseline Mean (SD) | 9.5 (3.16) | 9.0 (3.86) |
| Baseline Median | 10.0 | 9.0 |
| Change from baseline | | |
| Mean (SD) | −0.9 (1.25) | −2.1 (2.68) |
| Median | 0.0 | −2.0 |
| Range | (−3; 0) | (−8; 2) |
| IQ range | (−2.0; 0.0) | (−4.0; 0.0) |
| Percent change from baseline | | |
| Mean (SD) | −8.93 (13.139) | −23.74 (27.061) |
| Median | 0.00 | −22.50 |
| Range | (−33.3; 0.0) | (−75.0; 20.0) |
| IQ range | (−19.05; 0.00) | (−36.65; 0.00) |

A summary of change and percent change from baseline in S2K RI-50 over time are presented in Table 7. Overall, data from the ANTIBODY A cohort demonstrated numerically greater reduction that that from the placebo cohort: mean (SD) percentage change from baseline at day 100 for S2K RI-50: −51.77 (24.760) versus −25.84 (22.163), respectively.

TABLE 7
Summary of Change and Percent Change from Baseline in
S2K RI-50 Over Time in Part B

| | | ANTIBODY |
|---|---|---|
| | | A 10 mg/kg |
| | Placebo | IV |
| Analysis Set: Full Analysis Set | 8 | 18 |
| SLEDAI-2K RI-50 (0-105) | | |
| Baseline Mean (SD) | 9.3 (3.69) | 8.8 (3.70) |
| Baseline Median | 10.0 | 8.5 |
| Subjects evaluable at day 15 | 8 | 17 |
| Baseline Mean (SD) | 9.3 (3.69) | 9.0 (3.74) |
| Baseline Median | 10.0 | 9.0 |
| Change from baseline | | |
| Mean (SD) | −1.8 (4.20) | −3.2 (4.34) |
| Median | −2.0 | −3.0 |
| Range | (−6; 4) | (−14; 6) |
| IQ range | (−6.0; 2.0) | (−4.0; 0.0) |
| Percent change from baseline | | |
| Mean (SD) | 9.23 (86.610) | −29.71 (36.825) |
| Median | −20.00 | −33.30 |
| Range | (−60.0; 200.0) | (−70.0; 75.0) |
| IQ range | (−46.45; 33.35) | (−60.00; 0.00) |
| Subjects evaluable at Day 29 | 8 | 17 |
| Baseline Mean (SD) | 9.3 (3.69) | 9.0 (3.74) |
| Baseline Median | 10.0 | 9.0 |
| Change from baseline | | |
| Mean (SD) | −3.1 (2.36) | −3.6 (5.00) |
| Median | −2.0 | −2.0 |
| Range | (−7; 0) | (−16; 4) |
| IQ range | (−5.0; −2.0) | (−6.0; 0.0) |
| Percent change from baseline | | |
| Mean (SD) | −29.16 (17.342) | −30.52 (40.095) |
| Median | −26.65 | −25.00 |
| Range | (−50.0; 0.0) | (−83.3; 50.0) |
| IQ range | (−45.00; −20.00) | (−66.70; 0.00) |
| Subjects evaluable at Day 57 | 8 | 17 |
| Baseline Mean (SD) | 9.3 (3.69) | 9.0 (3.74) |
| Baseline Median | 10.0 | 9.0 |
| Change from baseline | | |
| Mean (SD) | −2.8 (4.27) | −5.1 (4.68) |
| Median | −2.0 | −6.0 |
| Range | (−8; 4) | (−18; 0) |
| IQ range | (−7.0; 0.0) | (−8.0; −2.0) |
| Percent change from baseline | | |
| Mean (SD) | −19.64 (43.872) | −47.25 (35.444) |
| Median | −20.00 | −50.00 |
| Range | (−66.7; 66.7) | (−100.0; 0.0) |
| IQ range | (−58.55; 0.00) | (−75.00; −20.00) |
| Subjects evaluable at Day 71 | 8 | 17 |
| Baseline Mean (SD) | 9.3 (3.69) | 9.0 (3.74) |
| Baseline Median | 10.0 | 9.0 |
| Change from baseline | | |
| Mean (SD) | −2.0 (3.66) | −5.9 (4.68) |
| Median | −2.5 | −6.0 |
| Range | (−6; 5) | (−20; 0) |
| IQ range | (−5.0; 0.0) | (−8.0; −2.0) |
| Percent change from baseline | | |
| Mean (SD) | −12.45 (42.994) | −58.45 (28.625) |
| Median | −25.00 | −60.00 |
| Range | (−50.0; 83.3) | (−100.0; 0.0) |
| IQ range | (−41.45; 0.00) | (−77.80; −33.30) |
| Subjects evaluable at day 100 | 8 | 18 |
| Baseline Mean (SD) | 9.3 (3.69) | 8.8 (3.70) |
| Baseline Median | 10.0 | 8.5 |
| Change from baseline | | |
| Mean (SD) | −2.9 (2.59) | −4.9 (3.52) |
| Median | −2.0 | −4.0 |
| Range | (−7; 0) | (−14; 0) |
| IQ range | (−5.0; −1.0) | (−7.0; −2.0) |
| Percent change from baseline | | |
| Mean (SD) | −25.84 (22.163) | −51.77 (24.760) |
| Median | −20.00 | −50.00 |
| Range | (−60.0; 0.0) | (−100.0; 0.0) |
| IQ range | (−45.00; −8.35) | (−70.00; −33.30) |

### PGA

A summary of change and percent change from baseline in PGA is presented in Table 8. Overall, data from the ANTIBODY A cohort demonstrated numerically greater reduction of PGA than that from the placebo cohort (mean[SD] percentage change from baseline at day 100: −19.60 (36.250) versus −6.09 (29.154), respectively).

TABLE 8
Summary of Change and Percent Change from Baseline in PGA in Part B

| | | ANTIBODY |
|---|---|---|
| | | A 10 mg/kg |
| | Placebo | IV |
| Analysis Set: Full Analysis Set | 8 | 18 |
| Physician's Global Assessment | | |
| (PGA VAS 0-10) | | |
| Baseline Mean (SD) | 3.00 (1.410) | 2.74 (1.300) |
| Baseline Median | 2.50 | 2.40 |
| Subjects evaluable at day 15 | 8 | 17 |
| Baseline Mean (SD) | 3.00 (1.410) | 2.68 (1.313) |
| Baseline Median | 2.50 | 2.30 |
| Change from baseline | | |
| Mean (SD) | −0.44 (0.933) | 0.02 (0.623) |
| Median | −0.35 | 0.00 |
| Range | (−2.0; 1.0) | (−1.0; 1.5) |
| IQ range | (−0.95; 0.05) | (−0.40; 0.20) |
| Percent change from baseline | | |
| Mean (SD) | −1.73 (44.697) | 4.48 (30.382) |
| Median | −11.01 | 0.00 |
| Range | (−50.0; 100.0) | (−33.3; 71.4) |
| IQ range | (−22.73; 1.83) | (−14.29; 14.29) |
| Subjects evaluable at Day 29 | 8 | 17 |
| Baseline Mean (SD) | 3.00 (1.410) | 2.68 (1.313) |
| Baseline Median | 2.50 | 2.30 |
| Change from baseline | | |
| Mean (SD) | −0.39 (1.236) | 0.15 (1.232) |
| Median | 0.05 | −0.10 |
| Range | (−3.1; 0.8) | (−1.0; 4.3) |
| IQ range | (−0.75; 0.30) | (−0.40; 0.50) |
| Percent change from baseline | | |
| Mean (SD) | −3.34 (46.204) | 12.42 (67.063) |
| Median | 0.91 | −4.76 |
| Range | (−77.5; 80.0) | (−43.5; 252.9) |
| IQ range | (−27.92; 12.38) | (−17.39; 16.67) |
| Subjects evaluable at Day 57 | 8 | 17 |
| Baseline Mean (SD) | 3.00 (1.410) | 2.68 (1.313) |
| Baseline Median | 2.50 | 2.30 |
| Change from baseline | | |
| Mean (SD) | −0.60 (1.664) | −0.61 (0.797) |
| Median | −0.80 | −0.50 |
| Range | (−3.9; 1.5) | (−2.2; 1.3) |
| IQ range | (−1.00; 0.60) | (−1.00; −0.10) |
| Percent change from baseline | | |
| Mean (SD) | −3.86 (66.434) | −18.72 (28.865) |
| Median | −21.59 | −22.73 |
| Range | (−97.5; 120.0) | (−48.3; 76.5) |
| IQ range | (−35.12; 30.00) | (−34.92; −9.52) |
| Subjects evaluable at Day 71 | 8 | 17 |
| Baseline Mean (SD) | 3.00 (1.410) | 2.68 (1.313) |
| Baseline Median | 2.50 | 2.30 |
| Change from baseline | | |
| Mean (SD) | −0.03 (1.430) | −0.42 (1.213) |
| Median | −0.50 | −0.60 |
| Range | (−1.5; 3.0) | (−2.1; 2.3) |
| IQ range | (−0.95; 0.60) | (−1.10; 0.10) |
| Percent change from baseline | | |
| Mean (SD) | 3.60 (46.142) | −3.77 (57.836) |
| Median | −18.61 | −26.09 |
| Range | (−37.5; 75.0) | (−62.5; 135.3) |
| IQ range | (−30.75; 45.00) | (−34.78; 2.86) |
| Subjects evaluable at day 100 | 8 | 18 |
| Baseline Mean (SD) | 3.00 (1.410) | 2.74 (1.300) |
| Baseline Median | 2.50 | 2.40 |
| Change from baseline | | |
| Mean (SD) | −0.40 (0.959) | −0.71 (1.060) |
| Median | −0.40 | −0.55 |
| Range | (−2.0; 1.0) | (−3.3; 1.5) |
| IQ range | (−1.00; 0.30) | (−1.00; −0.10) |
| Percent change from baseline | | |
| Mean (SD) | −6.09 (29.154) | −19.60 (36.250) |
| Median | −15.89 | −30.95 |
| Range | (−50.0; 40.0) | (−57.1; 100.0) |
| IQ range | (−21.82; 18.33) | (−39.13; −14.29) |

### Joint Assessment

A summary of change from baseline in the number of joints with pain and signs of inflammation over time for subjects with at least 2 affected joints at baseline is presented in Table 9.

TABLE 9
Summary of Change from Baseline in Number of Joints
with Pain and Signs of Inflammation Over Time for Subjects
with at Least 2 Affected Joints at Baseline in Part B

| | | ANTIBODY |
|---|---|---|
| | | A 10 mg/kg |
| | Placebo | IV |
| Analysis Set: Full Analysis Set | 8 | 18 |
| Subjects with at Least 2 Joints with | | |
| Pain and Signs of Inflammation | | |
| (Tenderness, Swelling or Effusion) at Baseline | 4 | 6 |
| Baseline Mean (SD) | 4.5 (3.00) | 2.3 (0.52) |
| Baseline Median | 3.0 | 2.0 |
| Change from Baseline in Number | | |
| of Joints with Pain and Signs of | | |
| Inflammation (Tenderness, | | |
| Swelling or Effusion) | | |
| Subjects evaluable at day 15 | 4 | 6 |
| Baseline Mean (SD) | 4.5 (3.00) | 2.3 (0.52) |
| Baseline Median | 3.0 | 2.0 |
| Change from baseline | | |
| Mean (SD) | −2.0 (1.41) | −1.0 (1.55) |
| Median | −2.5 | −1.5 |
| Range | (−3; 0) | (−2; 2) |
| IQ range | (−3.0; −1.0) | (−2.0; −1.0) |
| Subjects evaluable at Day 29 | 4 | 6 |
| Baseline Mean (SD) | 4.5 (3.00) | 2.3 (0.52) |
| Baseline Median | 3.0 | 2.0 |
| Change from baseline | | |
| Mean (SD) | 2.5 (7.05) | −1.7 (0.82) |
| Median | −0.5 | −2.0 |
| Range | (−2; 13) | (−2; 0) |
| IQ range | (−1.5; 6.5) | (−2.0; −2.0) |
| Subjects evaluable at Day 57 | 4 | 6 |
| Baseline Mean (SD) | 4.5 (3.00) | 2.3 (0.52) |
| Baseline Median | 3.0 | 2.0 |
| Change from baseline | | |
| Mean (SD) | −1.8 (0.50) | −1.2 (0.75) |
| Median | −2.0 | −1.0 |
| Range | (−2; −1) | (−2; 0) |
| IQ range | (−2.0; −1.5) | (−2.0; −1.0) |
| Subjects evaluable at day 71 | 4 | 6 |
| Baseline Mean (SD) | 4.5 (3.00) | 2.3 (0.52) |
| Baseline Median | 3.0 | 2.0 |
| Change from baseline | | |
| Mean (SD) | −1.3 (0.96) | −1.8 (0.41) |
| Median | −1.5 | −2.0 |
| Range | (−2; 0) | (−2; −1) |
| IQ range | (−2.0; −0.5) | (−2.0; −2.0) |
| Subjects evaluable at day 100 | 4 | 6 |
| Baseline Mean (SD) | 4.5 (3.00) | 2.3 (0.52) |
| Baseline Median | 3.0 | 2.0 |
| Change from baseline | | |
| Mean (SD) | −1.3 (1.71) | −1.2 (1.60) |
| Median | −1.5 | −2.0 |
| Range | (−3; 1) | (−2; 2) |
| IQ range | (−2.5; 0.0) | (−2.0; −1.0) |
| Subjects with at Least 2 Joints with | 4 | 6 |
| Tenderness (pain) at Baseline | | |
| Change from Baseline in Number | | |
| of Joints with Tenderness (pain) | | |
| Subjects evaluable at day 15 | 4 | 6 |
| Baseline Mean (SD) | 4.5 (3.00) | 2.3 (0.52) |
| Baseline Median | 3.0 | 2.0 |
| Range IQ range | (−8; 17) | (−11; 2) |
| Subjects evaluable at day 57 | (−5.5; 8.0) | (−3.0; −2.0) |
| | 4 | 9 |
| Baseline Mean (SD) | 7.5 (4.36) | 9.9 (6.57) |
| Baseline Median | 6.5 | 9.0 |
| Change from baseline | | |
| Mean (SD) | −2.8 (4.50) | −4.4 (3.61) |
| Median | −1.5 | −3.0 |
| Range | (−9; 1) | (−10; 0) |
| IQ range | (−6.0; 0.5) | (−8.0; −2.0) |
| Subjects evaluable at day 71 | 4 | 9 |
| Baseline Mean (SD) | 7.5 (4.36) | 9.9 (6.57) |
| Baseline Median | 6.5 | 9.0 |
| Change from baseline | | |
| Mean (SD) | −1.8 (6.99) | −5.2 (6.91) |
| Median | −2.0 | −2.0 |
| Range | (−10; 7) | (−20; 2) |
| IQ range | (−6.5; 3.0) | (−5.0; −2.0) |
| Subjects evaluable at day 100 | 4 | 9 |
| Baseline Mean (SD) | 7.5 (4.36) | 9.9 (6.57) |
| Baseline Median | 6.5 | 9.0 |
| Change from baseline | | |
| Mean (SD) | −2.0 (2.45) | −4.9 (4.01) |
| Median | −1.5 | −4.0 |
| Range | (−5; 0) | (−12; 1) |
| IQ range | (−4.0; 0.0) | (−7.0; −2.0) |

A summary of change from baseline in the number of joints with pain and signs of inflammation over time is presented in Table 10.

TABLE 10
Summary of Change from Baseline in Number of
Joints with Pain and Signs of Inflammation Over Time in Part B

| | | ANTIBODY |
|---|---|---|
| | | A 10 mg/kg |
| | Placebo | IV |
| Analysis Set: Full Analysis Set | 8 | 18 |
| Subjects evaluable for Joints | 8 | 18 |
| with Pain and Signs of Inflammation | | |
| (Tenderness, Swelling or Effusion) at Baseline | | |
| Baseline Mean (SD) | 2.8 (2.71) | 1.2 (0.94) |
| Baseline Median | 2.0 | 1.0 |
| Change from Baseline in Number | | |
| of Joints with Pain and Signs of | | |
| Inflammation (Tenderness, | | |
| Swelling or Effusion) | | |
| Subjects evaluable at day 15 | 8 | 17 |
| Baseline Mean (SD) | 2.8 (2.71) | 1.2 (0.97) |
| Baseline Median | 2.0 | 1.0 |
| Change from baseline | | |
| Mean (SD) | −1.0 (1.41) | −0.4 (1.00) |
| Median | 0.0 | 0.0 |
| Range | (−3; 0) | (−2; 2) |
| IQ range | (−2.5; 0.0) | (−1.0; 0.0) |
| Subjects evaluable at day 29 | 8 | 17 |
| Baseline Mean (SD) | 2.8 (2.71) | 1.2 (0.97) |
| Baseline Median | 2.0 | 1.0 |
| Change from baseline | | |
| Mean (SD) | 1.3 (4.80) | −0.6 (0.93) |
| Median | 0.0 | 0.0 |
| Range | (−2; 13) | (−2; 0) |
| IQ range | (−0.5; 0.0) | (−2.0; 0.0) |
| Subjects evaluable at day 57 | 8 | 17 |
| Baseline Mean (SD) | 2.8 (2.71) | 1.2 (0.97) |
| Baseline Median | 2.0 | 1.0 |
| Change from baseline | | |
| Mean (SD) | −0.9 (0.99) | −0.4 (0.71) |
| Median | −0.5 | 0.0 |
| Range | (−2; 0) | (−2; 0) |
| IQ range | (−2.0; 0.0) | (−1.0; 0.0) |
| Subjects evaluable at day 71 | 8 | 17 |
| Baseline Mean (SD) | 2.8 (2.71) | 1.2 (0.97) |
| Baseline Median | 2.0 | 1.0 |
| Change from baseline | | |
| Mean (SD) | −0.6 (0.92) | −0.6 (1.00) |
| Median | 0.0 | 0.0 |
| Range | (−2; 0) | (−2; 1) |
| IQ range | (−1.5; 0.0) | (−2.0; 0.0) |
| Subjects evaluable at day 100 | 8 | 18 |
| Baseline Mean (SD) | 2.8 (2.71) | 1.2 (0.94) |
| Baseline Median | 2.0 | 1.0 |
| Change from baseline | | |
| Mean (SD) | −0.8 (1.28) | −0.3 (1.08) |
| Median | −0.5 | 0.0 |
| Range | (−3; 1) | (−2; 2) |
| IQ range | (−1.5; 0.0) | (−1.0; 0.0) |
| Subjects with Tenderness (pain) at Baseline | 8 | 18 |
| Change from Baseline in Number | | |
| of Joints with Tenderness (pain) | | |
| Subjects evaluable at day 15 | 8 | 17 |
| Baseline Mean (SD) | 2.8 (2.71) | 1.2 (1.01) |
| Baseline Median | 2.0 | 1.0 |
| Change from baseline | | |
| Mean (SD) | −0.9 (1.36) | −0.4 (0.87) |
| Median | 0.0 | 0.0 |
| Range | (−3; 0) | (−2; 1) |
| IQ range | (−2.0; 0.0) | (−1.0; 0.0) |
| Subjects evaluable at day 29 | 8 | 17 |
| Baseline Mean (SD) | 2.8 (2.71) | 1.2 (1.01) |
| Baseline Median | 2.0 | 1.0 |
| Change from baseline | | |
| Mean (SD) | 1.8 (6.20) | −0.6 (0.94) |
| Median | 0.0 | 0.0 |
| Range | (−2; 17) | (−2; 0) |
| IQ range | (−0.5; 0.0) | (−2.0; 0.0) |
| Subjects evaluable at day 57 | 8 | 17 |
| Baseline Mean (SD) | 2.8 (2.71) | 1.2 (1.01) |
| Baseline Median | 2.0 | 1.0 |
| Change from baseline | | |
| Mean (SD) | −1.5 (2.07) | −0.4 (0.79) |
| Median | −1.0 | 0.0 |
| Range | (−6; 0) | (−2; 1) |
| IQ range | (−2.0; 0.0) | (−1.0; 0.0) |
| Subjects evaluable at day 71 | 8 | 17 |
| Baseline Mean (SD) | 2.8 (2.71) | 1.2 (1.01) |
| Baseline Median | 2.0 | 1.0 |
| Change from baseline | | |
| Mean (SD) | −0.9 (0.99) | 0.4 (3.92) |
| Median | −0.5 | 0.0 |
| Range | (−2; 0) | (−2; 15) |
| IQ range | (−2.0; 0.0) | (−2.0; 0.0) |
| Subjects evaluable at day 100 | 8 | 18 |
| Baseline Mean (SD) | 2.8 (2.71) | 1.2 (0.99) |
| Baseline Median | 2.0 | 1.0 |
| Change from baseline | | |
| Mean (SD) | −1.5 (2.56) | −0.3 (1.13) |
| Median | −0.5 | 0.0 |
| Range | (−7; 1) | (−2; 2) |
| IQ range | (−2.5; 0.0) | (−1.0; 0.0) |
| Subjects with Swelling or Effusion | 8 | 18 |
| at Baseline Change from Baseline | | |
| in Number of Joints with Swelling or | | |
| Effusion | | |
| Subjects evaluable at day 15 | 8 | 17 |
| Baseline Mean (SD) | 4.3 (4.50) | 5.6 (6.57) |
| Baseline Median | 2.5 | 3.0 |
| Change from baseline | | |
| Mean (SD) | −1.9 (3.83) | −0.6 (5.03) |
| Median | 0.0 | 0.0 |
| Range | (−11; 0) | (−16; 8) |
| IQ range | (−2.0; 0.0) | (−1.0; 2.0) |
| Subjects evaluable at day 29 | 8 | 17 |
| Baseline Mean (SD) | 4.3 (4.50) | 5.6 (6.57) |
| Baseline Median | 2.5 | 3.0 |
| Change from baseline | | |
| Mean (SD) | 0.6 (7.17) | −1.5 (2.96) |
| Median | 0.0 | 0.0 |
| Range | (−8; 17) | (−11; 2) |
| IQ range | (−2.0; 0.0) | (−2.0; 0.0) |

Overall, data from the ANTIBODY A cohort showed greater reduction in the number of swollen joints than that from the placebo cohort.

### Flares

A summary of time to first SLEDAI, severe SLEDAI or BILAG flare from day 1 through day 100 is presented in Table 6 above. Due to the small number of total flares comparisons between the cohorts were difficult; however, there were no notable differences between the treatment groups.

### CLASI

Low baseline activity scores rendered interpretation difficult. Judging by the percentage of subjects who experienced a 3-point deduction or 18% deduction from baseline at day 100 (defined as change in CLASI score that corresponds to clinical improvement), the ANTIBODY A cohort showed greater response than the placebo cohort (44.4% vs 25.0%, respectively).

### EQ-5D-5L and SF-36

No changes were evident in the overall scores or individual domains across the cohorts.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present inventions as defined by the specific description.

1. A method of administering an anti-IFN-α/-ω antibody to a human subject in need thereof in a clinically proven safe amount, comprising subcutaneously or intravenously administering to the subject a pharmaceutical composition comprising the antibody and a pharmaceutically acceptable carrier, wherein the antibody comprises a heavy chain variable region and a light chain variable region, the heavy chain variable region comprising heavy chain complementarity determining regions (HCDRs) HCDR1, HCDR2 and HCDR3 amino acid sequences of SEQ ID NOs: 33, 34, and 35, respectively, and the light chain variable region comprising light chain complementarity determining regions (LCDRs) LCDR1, LCDR2 and LCDR3 amino acid sequences of SEQ ID NOs: 30, 31, and 32, respectively, and wherein a total dosage of the antibody administered is 0.1 mg/kg to 20 mg/kg body weight of the subject per administration.

2. The method of claim 1, wherein the antibody comprises a heavy chain variable region (VH) having the amino acid sequence of SEQ ID NO: 28 and a light chain variable region (VL) having the amino acid sequences of SEQ ID NO: 29.

3. The method of claim 1, wherein the pharmaceutical composition is administered intravenously and the total dosage of the anti-IFN-α/-ω antibody administered per administration is 0.1 mg/kg, 0.3 mg/kg, 1 mg/kg, 3 mg/kg, 10 mg/kg, 15 mg/kg or 20 mg/kg body weight of the subject, or any dosage in between.

4. The method of claim 1, wherein the pharmaceutical composition is administered subcutaneously and the total dosage of the anti-IFN-α/-ω antibody administered per administration is 0.1 mg/kg, 0.3 mg/kg, 0.5 mg/kg, 0.7 mg/kg, 1 mg/kg, 1.2 mg/kg, 1.5 mg/kg, 1.7 mg/kg, 2 mg/kg, 2.3 mg/kg or 2.5 mg/kg body weight of the subject, or any dosage in between.

5. The method of claim 1, wherein the method achieves, in the plasma of the subject, at least one parameter selected from: (i) an area under the concentration time curve (AUC)₍₀₋ₜ₎ of about 50 μg.day/mL to about 7000 μg.day/mL, and (ii) a maximum concentration observed (Cₘₐₓ) of about 5 μg/mL to about 500 μg/mL.

6. The method of claim 1, wherein the administering of the anti-IFN-α/-ω antibody does not result in production of antibodies against the anti-IFN-α/-ω antibody in the subject.

7. The method of claim 1, wherein the human subject is in need of a treatment of an IFN-I mediated disease, such as systemic lupus erythematosus (SLE), type I diabetes, psoriasis, primary Sjögren's disease, systemic sclerosis, rheumatoid arthritis, transplant rejection, dermatomyositis, polymyositis, Aicardi-Goutières syndrome, Sting associated vasculopathy with onset in infancy (SAVI) or chronic atypical neutrophilic dermatosis with lipodystrophy and elevated temperature syndrome (CANDLE).

8. The method of claim 7, wherein the human subject is in need of a treatment of mild to moderate systemic lupus erythematosus (SLE).

9. The method of claim 8, wherein the pharmaceutical composition is administered to the human subject intravenously for no less than 30 minutes in a total dosage of the anti-IFN-α/-ω antibody administered of 10 mg/kg body weight of the subject per administration, preferably the pharmaceutical composition is intravenously administered to the human subject repeatedly, more preferably once every two weeks.

10. The method of claim 8, wherein the pharmaceutical composition is administered to the human subject subcutaneously in a total dosage of the anti-IFN-α/-ω antibody administered of 1 mg/kg body weight of the subject per administration.

11. The method of claim 8, wherein the administering of the pharmaceutical composition achieves, in the plasma of the subject, at least one parameter selected from: (i) an area under the concentration time curve (AUC)_{(0-14d)} of about 1000 μg.day/mL to about 3500 μg.day/mL, and (ii) a maximum concentration observed (Cₘₐₓ) of about 120 μg/mL to about 400 μg/mL.

12. The method of claim 8, wherein the human subject has a reduction in the Systemic Lupus Erythematosus Responder Index (SRI), preferably a reduction of 4 points, more preferably 5 points, and most preferably 6 points, by day 100 after the administration of the pharmaceutical composition.

13. The method of claim 8, wherein the human subject has no new British Isles Lupus Assessment Group (BILAG) A or 2B shifts by day 100 after the administration of the pharmaceutical composition.

14. The method of claim 8, wherein the human subject has a reduction in the Systemic Lupus Erythematosus Disease Activity Index 2000 (SLEDAI-2K) from baseline by day 100 after the administration of the pharmaceutical composition; and/or
wherein the human subject has a reduction in the Systemic Lupus Erythematosus 2000 Responder Index-50 (S2K RI-50) from baseline by day 100 after the administration of the pharmaceutical composition; and/or
wherein the human subject has a reduction in the Physician's Global Assessment of Disease Activity (PGA) from baseline by day 100 after the administration of the pharmaceutical composition.

15. (canceled)

16. (canceled)

17. The method of claim 8, wherein the human subject achieves a steady-state condition of the antibody within 40-50 days after administration.

18. The method of claim 8, wherein the administering of the anti-IFN-α/-ω antibody does not result in a treatment emergent adverse event (TEAE) related to a malignancy or anaphylactic or serum sickness-type reaction in the subject.

19. The method of claim 8, further comprising:
a. assaying gene expression of one or more genes DHX58, EIF2AK2, HERC5, IFI44, IFI44L, IFI6, IRF7, PARP9, PLSCR1 and SAMD9L in a biological sample of the human subject; and
b. identifying the human subject as responsive to treatment of the antibody prior to administering to the subject the pharmaceutical composition comprising the antibody and the pharmaceutically acceptable carrier.

20. The method of claim 19, comprising:
a. assaying gene expression of genes DHX58, EIF2AK2, HERC5, IFI44, IFI44L, IFI6, IRF7, PARP9, PLSCR1 and SAMD9L in a biological sample of the human subject;
b. determining a combined expression value of the genes DHX58, EIF2AK2, HERC5, IFI44, IFI44L, IFI6, IRF7, PARP9, PLSCR1 and SAMD9L in the biological sample; and
c. identifying the human subject as responsive to treatment of the antibody when the combined expression value is equal to or higher than a threshold value.

21. The method of claim 8, wherein the human subject has been treated with chloroquine, hydroxychloroquine, methotrexate or systemic corticosteroids, or any combination thereof; or
further comprising administering chloroquine, hydroxychloroquine, methotrexate or systemic corticosteroids, or any combination thereof to the human subject.

22. (canceled)

23. The method of claim 8, wherein the anti-IFN-α/-ω antibody is administered in a formulation selected from the group consisting of:
30 μg/mL of the anti-IFN-α/-ω antibody, 5 mM lactate, 5% (w/v) lactose, and 0.02% (w/v) polysorbate 80 (PS80), at pH 5.5;
40 mg/mL of the anti-IFN-α/-ω antibody, 8 mM acetate, 8% (w/v) maltose, and 0.06% (w/v) polysorbate 20 (PS20), at pH 5.0;
50 mg/mL of the anti-IFN-α/-ω antibody, 13 mM acetate, 8.0% (w/v) sucrose, and 0.04% (w/v) polysorbate 20 (PS20), at pH 5.2;
60 mg/mL of the anti-IFN-α/-ω antibody, 10 mM acetate, 8% (w/v) lactose, and 0.06% (w/v) polysorbate 20 (PS20), at pH 5.4; and
70 mg/mL of the anti-IFN-α/-ω antibody, 15 mM lactate, 10% (w/v) maltose, and 0.06% (w/v) polysorbate 80 (PS80), at pH 5.0.
